(19) United States
(12) Patent Application Publication
(10) Pub. No.: US 2015/0033371 A1
(43) Pub. Date: Jan. 29, 2015

(54) HAIR FOLLICLE STEM CELLS AND METHODS OF USE SAME

(71) Applicants: Krzysztof Kobielak, San Gabriel, CA (US); Eve Kandyba, Pasadena, CA (US)

(72) Inventors: Krzysztof Kobielak, San Gabriel, CA (US); Eve Kandyba, Pasadena, CA (US)

(73) Assignee: University of Southern California, Los Angeles, CA (US)

(21) Appl. No.: 14/344,898

(22) PCT Filed: Sep. 13, 2012
(86) PCT No.: PCT/US2012/055217

§ 371 Date: Sep. 18, 2014

Related U.S. Application Data
(60) Provisional application No. 61/534,828, filed on Sep. 14, 2011.
(60) Provisional application No. 61/539,936, filed on Sep. 27, 2011.

(51) Int. Cl. C12N 5/071; A01K 67/027; G01N 33/50; A61K 35/36

(52) U.S. Cl. CPC C12N 5/0628; A61K 35/36; A01K 67/0275; G01N 33/5073; A01K 2267/0393; A01K 2227/105; G01N 2500/10
USPC 800/13; 424/93.21; 435/325; 435/29; 435/6.1; 435/375

(57) ABSTRACT

The disclosure provides isolated hair follicle stem cells (hfSC) having inactivated BMP signaling, which are discovered to express keratin 15. Also provided are hfSC that have constitutively activated BMP signaling and methods of using such hfSC for screening candidate agents suitable for inhibiting BMP signaling in hfSC and promoting hair growth. Further provided are methods of activating hfSC and promoting hair growth from hfSC.

### CROSS-REFERENCE TO RELATED APPLICATIONS

This application claims the benefit under 35 U.S.C. §119(e) of U.S. Provisional Application Ser. Nos. 61/534,828, filed Sep. 14, 2011, and 61/539,936, filed Sep. 27, 2011, the content of each of which is incorporated by reference in its entirety into the present disclosure.

### BACKGROUND

During the hair cycle, the behavior of slow-cycling, hair follicle stem cells (hfSCs) is tightly governed by an intricate balance of signaling pathways which converge to induce bouts of SC quiescence and activation, resulting in new hair formation. It remains to be understood how this regenerative hair cycling behavior is regulated at the molecular level, and to apply this knowledge toward regenerative medicine.

Bone morphogenetic protein (BMP) signaling is known to be important for hair follicle stem cells homeostasis. Ablation of the Bmpr1a gene resulted in the precocious activation of quiescent hfSCs, suggesting BMP activity plays an inhibiting role in hfSC homeostasis. However, how BMP signaling integrates different activators and inhibitors to achieve a molecular network capable of cyclic activation of hfSCs is not known.

Although hair follicle stem cells are one of the best-characterized adult stem cell systems, it still is not known how BMP signaling integrates different regulators into a molecular network capable of cyclic activation of hfSCs. The main obstacle to address this important question in stem cell biology was due to the loss of expression of CD34, the only available marker for isolation of hfSCs upon BMP inhibition.

### SUMMARY

Previous studies revealed the importance of balancing BMP signaling in hair follicle stem cell (hfSC) regulation however, the precise molecular mechanism of BMP signaling functions remains unknown since loss of expression of the only available marker, CD34, is available for isolation of hfSCs. To overcome this problem, genetic in vivo strategies were developed that allow the isolation, characterization and culture of hfSCs cells where BMP signaling was inactivated specifically in hfSC. The transcription profile of hfSCs after inactivation of BMP signaling reveals that hfSCs switch from quiescence to activation and acquire molecular characteristics resembling the hair germ.

Interestingly Applicants found that hfSCs with suppressed BMP signaling show altered expression in the BMP pathway itself and in Wnt pathway, demonstrating upregulation of certain Wnt ligands which correlated with upregulation of the only one of Wnt receptor. Together, it can show the crosstalk mechanism where inhibition of BMP signaling in hfSCs is crucial to activate downstream effectors of WNT signaling in ligand dependent manner.

The present disclosure, for the first time, discloses isolated hair follicle stem cells (hfSC) having inactivated BMP signaling owing to the discovery that such hfSC express keratin 15. Using these isolated hfSC, the present disclosure identified genes that are up- or down-regulated upon inhibition of BMP signaling. Inducible hfSC in which BMP signaling can be constitutively activated are also generated, providing a system for screening for agents suitable for activating hair germ cells. Further, it was discovered that, upon normalization of BMP signaling, new hair growth is reinitiated, demonstrating the effectiveness of promoting hair growth via BMP signaling normalization.

The disclosure thus provides isolated hair follicle stem cells (hfSC) having inactivated BMP signaling, which are discovered to express keratin 15. Also provided are hfSC that have constitutively activated BMP signaling and methods of using such hfSC for screening candidate agents suitable for inhibiting BMP signaling in hfSC and promoting hair growth. Further provided are methods of activating hfSC and promoting hair growth from hfSC.

Thus, in one embodiment, the present disclosure provides an isolated hair follicle stem cell (hfSC) that expresses keratin 15 (K15) and has decreased BMP signaling. In one aspect, the hfSC has decreased biological activity of BMPR1A. In another aspect, the BMPR1A is inactivated. In yet another aspect, the hfSC does not express CD34.

In one embodiment, the hfSC further comprises an exogenous reporter gene. In one aspect, the reporter gene is activated by a K15 promoter. In another aspect, the reporter gene is activated by a gene downstream of the K15 promoter. The activation can be mediated by a pharmaceutical agent that is in contact with the hfSC.

In another embodiment, the hfSC has increased expression of one or more of Wnt7a, Wnt7b, or Fzd10 and/or decreased expression of Dkk3 each as compared to a hfSC having normal BMP signaling.

Also provided, in one embodiment, is a population of the isolated hfSC having the characteristics described herein. In one aspect, the population is substantially homogenous. Still further provided, in one embodiment, is a substantially homogenous population of cells differentiated from an isolated hfSC having the characteristics provided herein.

Pharmaceutical compositions are also provided, comprising an isolated hfSC or a cell population of any of the above embodiments, and a pharmaceutically acceptable carrier.

In another embodiment, the present disclosure provides a method of treating alopecia in a mammalian subject, comprising implanting to the subject an isolated hair follicle stem cell (hfSC), a cell population, or a pharmaceutical composition of any of the above embodiments.

The present disclosure, in another embodiment, provides an isolated hair follicle stem cell (hfSC) comprising a constitutively active BMPR1A gene. In one aspect, the BMPR1A gene is regulated by a promoter inducible by a pharmaceutical agent that is in contact with the hfSC. In another aspect, the promoter is double Tg (dTg). In yet another aspect, the pharmaceutical agent is doxcycline. In some aspects, the isolated hfSC further comprises a reporter gene regulated by a keratin 15 (K15) promoter.

Also provided, in one embodiment, is a population of such isolated hfSC. In one aspect, the population is substantially homogenous. Another embodiment provides a clonal population of the isolated hfSC.

The present disclosure provides a transgenic non-human mammal comprising a hair follicle stem cell (hfSC) comprising a constitutively active BMPR1A gene. In some aspects, the hfSC further comprises a reporter gene regulated by a keratin 15 (K15) promoter.

Methods of identifying an agent suitable for inhibiting BMP signaling in a hair follicle are also provided. In one embodiment, the method comprises contacting a candidate agent with an isolated hfSC comprising a constitutively active BMPR1A gene or a genetically engineered non-human mammal comprising a constitutively active BMPR1A gene, wherein expression of the reporter gene indicates that the candidate agent is suitable for inhibiting BMP signaling in a hair follicle. Such a method is also useful for identifying an agent suitable for activating a hair follicle stem cell (hfSC).

One embodiment of the present disclosure provides a method of activating a hair follicle stem cell (hfSC), comprising increasing the biological activity of one or more of Wnt7a, Wnt7b, or Fzd10 and/or decreasing the biological activity of Dkk3. In one aspect, increasing of the biological activity of one or more of Wnt7a, Wnt7b, or Fzd10 comprises increasing of transcription of one or more of Wnt7a, Wnt7b, or Fzd10. In another aspect, the decreasing of the biological activity of Dkk3 comprises decreasing the transcription of Dkk3. In another aspect, the method further comprises contacting the hfSC with an agent that inhibits BMP signaling.

Methods of treating alopecia in a mammalian subject are provided, comprising implanting to the subject an activated a hair follicle stem cell (hfSC), wherein the hfSC was isolated from the subject and activated by a method as disclosed above.

### BRIEF DESCRIPTION OF THE FIGURES

FIG. 1 shows SMP signaling in hfSC homeostasis and that the hfSC marker, CD34, is lost when BMP signaling is inhibited. Upper panel: BMP inhibition results in loss of hfSCs marker, CD34 in hfSC; middle panel, FACS analysis confirms a decrease of CD34 expression in KO SCs; bottom panel, slow cycling, label-retaining cells are decreased after BMPR1a deletion, indicating expansion of the SC niche.

FIGS. 2A through 2C illustrate the generation of conditional inducible knockout mice, to specifically label and inactivate BMP signaling in hfSCs. (A), Schematics depict matings between three different lines of mice: K15-CrePR×BMPR1A flox/flox (FL/FL)×Rosa26-Stop-YFP(FL/FL). (B and C), Genotype of offspring from these matings: Con^{RU} means K15CrePR/BMPR1A WT/WT/YFP (FI/WT) and cKO^{RU} indicates K15CrePR/BMPR1A FI/FI/YFP (FI/WT). (D and E) Phenotype of adult Con^{RU} and cKO^{RU} mice, respectively after RU treatment. cKO^{TM} mice did not regrow hair. Abbreviations: Con^{RU}—control and cKO^{RU}—conditional knockout both after RU treatment at postnatal day 120 (P120); floxP and FL—loxedP side; Cre and Cre-PR—recombinase conjugated with truncuted progesterone receptor; YFP—yellow fluorescent protein.

FIGS. 3A through 3E show specific activation of the yellow fluorescent protein (YFP) in the hair follicle stem cells in BMPR1A inducible conditional knockout mouse. (A), Chart illustrating the temporal progression through the first and second postnatal hair cycles, and regiment for administering RU486 (RU) and time point the samples were collected for FACS (Ai) cKO^{RU} and CON^{RU} mice at (P59) the end of RU treatment (B), Whole back skin view from dermis (under stereomicroscope with YFP filter) from cKO^{RU} and CON^{RU} mice showing specific activation of YFP in the majority of hair follicle stem cells marked by arrows after 16 d of RU treatment (C), Higher magnification of bulges from cKO^{RU} and CON^{RU} (D), Sections through the single hair follicle from cKO^{RU} and CON^{RU} showing both hairs are still in telogen—regular DIC (E), the same section as C, showing YFP positive bulge, both in telogen. Abbreviation: Bu—bulge, RU—RU486.

FIGS. 4A through 4D show the isolation of YFP labelled hair follicle stem cells after inhibition of BMP signaling. To isolate Bmpr1a WT and KO hfSCs, YFP expression was induced (arrowheads) within the keratin-15+ bulge (Bu) region of both Bmpr1a WT (A) and Bmpr1a KO (E) hair follicles by topical RU486 application; FACS analysis showed approximately 1-2% of the total cell population isolated from WT (B) or KO (F) back skin was YFP+. By focusing upon the YFP+ (keratin-15+) fraction and by employing α6-integrin and CD34 FACS analysis, both WT (C) and KO (G) YFP+ fractions could be further categorized into three distinct subpopulations: 1) YFP+α6+; 2) YFP+CD34+ and 3) YFP+α6+ CD34+. Following Bmpr1a deletion, a considerable decrease in expression of the hair follicle stem cell marker, CD34 was observed in Bmpr1a KO YFP+ cells, notably in the CD34 high fractions (H) when compared with the same Bmpr1a WT YFP+CD34 high fractions (D).

FIGS. 5A through 5E show that microarray profiling reveals that YFP positive Bmpr1a KO hfSCs acquire a molecular characteristic towards that of the hair germ The YFP+ basal hfSC populations of α6-integrin and CD34 by were sorted by FACS from both Bmpr1a WT (A) and KO mice (B) and were used for microarray analysis; loxP recombination efficiency for exon 2 deletion in the Bmpr1a KO hfSCs population was confirmed by RT-PCR(C); Transcriptional profiling reveals down-regulation of 103 genes (green circle) out of 427 probes of commonly up-regulated hfSCs signature genes (gray circle) after inhibition of BMP signaling in hfSCs (D); comparison of hfSCs with inactivated BMP signaling with the hair germ signature genes. Approximately 30% of genes overlapped with the previously characterized hair germ signature (E).

FIG. 6 shows that the hair germ marker, P-cadherin, is expanded in activated KO hf SCs. The hair germ (hg) arises from the bulge (bu) at the end of catagen and is found at hair follicle base in contact with the dermal papillae (dp). At the end of telogen, the hair germ is activated by the dp before the bulge SCs. In vitro, hair germ cells proliferate quickly but exhaust their proliferative potential. HG prevents depletion of the hfSCs and enables rapid initial hair growth in vivo.

FIG. 7 shows that Bmpr1a KO YFP+ hfSCs develop hair germ characteristics in vivo but maintain SC-like potential in vitro. Like hg cells, Bmpr1a KO hfSCs proliferate faster in vitro but unlike hg cells, both YFP+ WT and KO hfSCs can be passaged many times (>P20) in vitro.

FIG. 8 shows a list of genes whose expressions were affected by Bmpr1a KO and it reveals that hfSCs BMP signaling affects both the BMP and WNT signaling pathway.

FIG. 9 illustrates a contemplated mechanism of a constant competitive cycling between activator and inhibitor activities in hfSCs population within the hair bulge

FIGS. 10A through 10I show that isolated YFP labeled Bmpr1a KO hfSCs maintain stem cell characteristics both in vitro and in vivo. Bmpr1a WT and KO hfSCs were isolated by FACS and cultured separately upon mitomycin-treated 3T3 fibroblast feeder layers resulting in the formation of discrete Bmpr1a WT (A) and KO (B) YFP+ stem cell colonies. Both YFP+ Bmpr1a WT and KO hfSCs could be passaged multiple times (>20 passages) in vitro. To validate the multipotentcy of hfSCs, each YFP+ cell population (at passage 4) was prepared for chamber graft on athymic mice (C) by mixing these cells with freshly isolated dermal fibroblasts (C′). In vivo chamber graft experiments were performed using the YFP+ Bmpr1a WT and KO cell suspensions, respectively, and at 8 weeks, following removal of the chamber dome, YFP+ expression was observable in both control (D) and KO (E) graft areas indicating survival of the hfSCs. Upon grafting, WT YFP+ hfSCs could regenerate new hair (F), however, Bmpr1a KO YFP+ hfSCs grafts (G) did not display any visible hair growth. Analysis of grafts by cryosection revealed that YFP+ Bmpr1a WT hfSCs retained the capacity to form an organized epidermis (dashed line) and reconstitute YFP+ hair follicles in vivo (H), whereas, YFP+ Bmpr1a KO YFP+ hfSCs instead formed an epidermis (dashed line) and malformed hair follicle structures that lacked a differentiated hair shaft (I).

FIGS. 11A through J show that BMP inactivation in hfSCs results in overexpression of WNT activator 2 (WNT7a) in the bulge and hair germ and nuclear beta-catenin stabilization. Physiological WNT activator 2 expression is observed in the bulge and hair germ during telogen to anagen transition and Beta-catenin stabilization. BMP activation, on the other hand, results in inhibition of WNT activator 2 expression in hf SCs.

FIG. 12 shows that subcutaneous injection of WNT activator 2 recombinant protein induces precocious hair germ activation.

FIG. 13 shows that subcutaneous injection of WNT activator 2 recombinant protein promotes precocious telogen to anagen transition.

FIG. 14 shows that Wnt Activator 3 (WNT7b) is expressed physiologically during the telogen-anagen transition.

FIG. 15 shows that Wnt Activator 3 deletion delays hair cycling.

FIG. 16 shows that during hair follicle morphogenesis, Wnt Activator 3 deletion delays but does not prevent hair growth.

FIG. 17 is a schematic of modified pSLIK-mCherry lentiviral gene delivery platform. The pEN_T vector with incorporated mCherry-internal ribosome entry site (IRES) technology (Clontech) with a multi-cloning site (MCS) for the gene of interest (GOI). Individual candidate BMP target GOI's will then be subcloned into the mCherry multi-cloning site (MCS). Individual modified mCherry-GOI constructs will then be recombined into the p-SLIK vector using site specific recombination. This doxycycline (DOXY)-inducible lentiviral system (tet-on) will reliably enable tight regulation of BMP target GOI expression in the hfSC populations in the presence of doxycycline (both in vitro and in vivo) while simultaneously labeling targeted cells with the fluorescent reporter, mCherry, to facilitate screening of cell populations (as already tested).

FIGS. 18A through 18G show that to identify in vivo candidate BMP signaling target genes in hfSCs, doxycycline-inducible dTg-GFP+ mice were engineered to express a constitutively active form of Bmpr1a. Mice were mated to be doubly transgenic (dTg) for the TRE-Bmpr1a-CA and K14rtTA, encoding a DOXY-inducible transactivator. To facilitate isolation of dTg hf SCs following oral administration of DOXY, dTg mice were also mated with K15-GFP mice (A). To assess the early implications of sustained BMP signaling, DOXY food was provided at P18 (B) and alkaline phosphatase staining was used to indicate morphological features of control (C, E) and dTg hair follicles (D, F). K15-GFP+ hf SCs were examined at specific time points (P18, P21, P25) during the first postnatal hair cycle and it was observed that, in contrast to control mice, dTg hair follicles did not enter full anagen by P25 but retained a K15-GFP+ bulge region. Using FACS sorting to isolate GFP+ hfSCs, it was shown that DOXY treatment (1 ug/ml) of hfSCs (G) can induce Bmpr1a transcript expression in vitro. This also demonstrated that, following oral administration of DOXY (for 7 days) in vivo, phospho-smad levels are increased within the dTg GFP+ hfSC population (H) in comparison to control samples.

FIGS. 19A through 19J show the re-establishment of new hair cyclic regeneration. Mice were engineered upon a K15-GFP background to be doubly transgenic (dTg) for TRE-Bmpr1a-CA, a tetracycline (DOXY) regulatable transgene encoding a constitutively active form of Bmpr1a and K14rtTA (encoding a DOXY-inducible transactivator). Following DOXY induced activation of BMP signaling at P18, dTg mice demonstrated hair growth inhibition and small cyst formation (D) and progressive hair loss (B), however, control mice (A, unresponsive to DOXY) displayed proper transition to the next hair cycle with new hair production (C). Control hair follicles retained bulge K15-GFP+ expression (E) but GFP+ expression was lost within the bulge of dTg-affected hair follicles and observed reorganization of the dermal papillae around the base of the cyst (F). Upon withdrawal of DOXY, dTg mice showed new hair production (H) to resemble control mice (G). The early re-organization events occur following DOXY withdrawal (removed at P65), biopsies were taken at 2 day intervals and hair follicle morphology was examined (I). Progressive thickening and down-growth from the cysts along with re-organization of the dermal papillae with condensing at the base of down-growth. 2-4 days after DOXY removal, GFP+ cells re-emerged within the cyst structures (J).

FIG. 20 shows that upon normalization of BMP signaling, new hair growth is initiated.

FIG. 21 illustrates a lineage tracing experiment.

FIG. 22 shows that mis-positioned dTg GFP+ cyst cells can initiate new hair growth following BMP normalization. GFP+/tdTomato cyst cells actively participate in new hair formation and can be isolated and cultured in vitro.

FIG. 23 illustrates the proposed BMP signaling regulating hair cell activation and growth based on the current data.

FIG. 24 illustrates that the current data indicate that in hf SCs, Bmpr1a acts through the Canonical BMP-Smad signaling pathway.

FIG. 25 shows that Smads 1, 5 and/or 8 are potentially involved in BMP signaling regulating hair cell activation and growth.

FIG. 26 shows that newborn Smad1 &5 dKO mice display eye, whisker and paw abnormalities and die within 24 hrs of birth.

FIG. 27 shows that Smad1 &5 dKO hair follicles are fewer and under-developed. Newborn Smad 1&5 dKO skin displays fewer and under-developed hair follicles than control skin.

FIG. 28 shows that Skin from Smad 1&5 dKO mice display normal epidermal differentiation but poorly developed (immature) hair follicles.

FIG. 29 shows that Smad 1&5 dKO skin transplants lack hair shaft formation. Smad1&5 deletion prevents normal hair formation indicating Smad8 acting alone cannot rescue loss-of-hair phenotype.

FIG. 30 show that, in vitro, Smad KO cell lines display altered cell morphology. These Smad cell lines can be used as a tool to help uncover which Smad proteins are significant in hf SC BMP signaling.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

### DEFINITIONS

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

“Bone Morphogenic Proteins” (BMP) are a group of multifunctional growth factors and cytokines with effects in various tissues. For example, BMPs are known to induce the formation of bone and/or cartilage. Examples of BMP may include, but are not limited to BMP1, BMP2, BMP3, BMP4, BMP5, BMP6, BMP7, BMP8a, BMP8b, BMP10 and BMP15.

“BMP signaling” or “BMP signaling pathway” refers to the enzyme linked receptor protein signaling transduction pathway involving proteins that directly or indirectly regulate (activate or inhibit) downstream protein activity or gene expression. Examples of molecules involved in the BMP signaling pathways may be found in the public Gene Ontology (GO) database, under GO ID: GO:0030509, accessible at the web page (amigo.geneontology.org/cgi-bin/amigo/term-details.cgi?term=GO:0030509&session_id=5573amigo1226631957), last accessed on Nov. 17, 2008. Without limitation, examples of proteins in the BMP signaling pathway include Activin receptor type-1 (ACVR1, UniProt: Q04771), Activin receptor type-2A (ACVR2A, UniProt: P27037), Activin receptor type-2B (ACVR2B, UniProt: Q13705), BMP1 (UniProt: P13497), BMP2 (UniProt: P12643), BMP3 (UniProt: P12645), BMP4 (UniProt: P12644), BMP5 (UniProt: P22003), BMP6 (UniProt: P22004), BMP7 (UniProt: P18075), BMP8a (UniProt: Q7Z5Y6), BMP8b (UniProt: P34820), BMP10 (UniProt: 095393), BMP15 (UniProt: 095972), Bone morphogenetic protein receptor type-1A (BMPR1A, UniProt: P36894), Bone morphogenetic protein receptor type-1B (BMPR1B, UniProt: 000238), Bone morphogenetic protein receptor type-2 (BMPR2, UniProt: Q13873), Chordin-like protein (CHRDL1, UniProt: Q9BU40), Follistatin-related protein 1 (FSTL1, UniProt: Q12841), Growth/differentiation factor 2 (GDF2, UniProt: Q9UK05), Growth/differentiation factor 6 (GDF6, UniProt: Q6KF10), Growth/differentiation factor 7 (GDF7, UniProt: Q7Z4P5), Gremlin-2 (GREM2, UniProt: Q9H772), RGM domain family member B (RGMB, UniProt: Q6NW40), Ski oncogene (SKI, UniProt: P12755), Mothers against decapentaplegic homolog 4 (SMAD4, UniProt: Q13485), Mothers against decapentaplegic homolog 5 (SMAD5, UniProt: Q99717), Mothers against decapentaplegic homolog 6 (SMAD6, UniProt: 043541), Mothers against decapentaplegic homolog 7 (SMAD7, UniProt: 015105), Mothers against decapentaplegic homolog 9 (SMAD9, UniProt: 015198), E3 ubiquitin-protein ligase SMRF2 (SMURF2, UniProt: Q9HAU4), TGF-beta receptor type III (TGFBR3, UniProt: Q03167), Ubiquitin-conjugating enzyme E2 D1 (UBE2D1, UniProt: P51668), Ubiquitin-conjugating enzyme E2 D3 (UBE2D3, UniProt: P61077) and Zinc finger FYVE domain-containing protein 16 (ZFYVE16, UniProt: Q7Z3T8). Proteins that positively or negatively regulate the BMP signaling, for purpose of this invention, are also considered within the meaning of the BMP signaling. Proteins that positively regulate BMP signaling include, but are not limited to, Serine/threonine-protein kinase receptor R3 (ACVRL1, UniProt: P37023) and Endoglin (ENG, UniProt: P17813). Proteins that negatively regulate BMP signaling include, but are not limited to, Chordin (CHRD, UniProt: Q9H2X0), E3 ubiquitin-protein ligase SMURF1 (SMURF1, UniProt: Q9HCE7), Sclerostin (SOST, UniProt: Q9BQB4) and Brorin (VWC2, UniProt: Q2TAL6). Examples of proteins in the BMP signaling pathway may also include Proprotein convertase subtilisin/kexin type 6 (PCSK6, UniProt: P29122) that regulates BMP signaling.

Small molecules, polynucleotides, polypeptides that enhance or inhibit BMP signaling exist or can be made with procedures known by those skilled in the art. For example, Dorsomorphin is a potent small molecule BMP antagonist (Hao et al. PLoS ONE, 2008, 3(8):e2904, Yu et al., Nat Chem Biol., 2008, 4(1):33-41). Dorsomorphin was reported to selectively inhibit the BMP receptors, type I: ALK2, ALK3 and ALK6 and thus “blocks BMP-mediated SMAD1/5/8 phosphorylation”. Dorsomorphin has preferential specificity toward inhibiting BMP vs. TGF-beta and activin signaling. In published reports, Dorsomorphin is characterized by low toxicity. Dorsomorphin is currently commercially available from several vendors. Dorsomorphin can be delivered into skin to lower macro-environmental BMP signaling and create favorable conditions for hair growth to occur. A unique property of Dorsomorphin is that it is a small molecule and is soluble in DMSO. DMSO is known to significantly facilitate transdermal delivery of small molecule drugs. This enhancing effect of DMSO on skin penetration can be used in non-invasive method of pharmacological modulation of dermal macro-environment. Treatment procedure thus consists of simply applying liquid form of Dorsomorphin in DMSO onto the surface of intact skin. Dorsomorphin in DMSO can be made in form of cream. Cream can be simply rubbed onto intact skin. Small molecule agonist and antagonists for other signaling pathways also exist and can be used to augment or inhibit BMP signaling. Interaction of these small molecules with pathways including, but not limited to, WNT, SHH and FGF will also have direct or indirect impact on BMP signaling thus serve as effective modulator of hair growth via methods disclosed in this invention.

Other types of BMP agonists or antagonists also exist. Non-limiting examples include such as noggin, chordin, gremlin, sclerostin and follistatin. Representative sequences for these proteins include UniProt: Q13253 for noggin, UniProt: Q9H2X0 for chordin, UniProt: 060565 for gremlin, UniProt: Q9BQB4 for sclerostin, and UniProt: P19883 for follistatin. Noggin (UniProt: Q13253), for example, can be produced using methods described in, e.g. McMahon et al. (1998) Genes & Development 12:1438-52.

In some aspects, an agent that can augment or inhibit BMP signaling is a small molecule agonist or antagonist to a BMP agonist or antagonist. In one aspect, the small molecule is a noggin agonist. In another aspect, the small molecule is a noggin antagonist.

Examples of agents that can augment or inhibit BMP signaling also include, but are not limited to, polynucleotides that encode BMP proteins, encode polypeptides augmenting or inhibiting BMP signaling, or augmenting or inhibit expression of BMP proteins, or polypeptides augmenting or inhibiting BMP signaling. In some embodiments, the agent is small interference RNA (siRNA) or double strand RNA (dsRNA) that inhibits expression of proteins that augment or inhibit BMP signaling.

Examples of agents that can augment or inhibit BMP signaling may also include, but are not limited to, an isolated or recombinant BMP protein, or isolated or recombinant polypeptide enhancing or inhibiting BMP signaling. In some aspect, the agent further comprises a pharmaceutically acceptable carrier. In another aspect, the compositions contain carriers that modulate (controlled release) the release of the active agent when administered to a subject in need thereof.

Examples of polypeptide agents that augment or inhibit BMP signaling may also include, but are not limited to, antibodies or modified antibodies including, but not limited to, blocking fragments of antibodies, that activate, stabilize or inhibit proteins in the BMP signaling pathway or proteins modulating the BMP signaling pathway, thereby augmenting or inhibiting BMP signaling.

The term “decreased BMP signaling” as used herein refers to a decrease in expression or activity of a BMP molecule whose activity is involved in carrying out the BMP signaling. In one aspect, the BMP molecule is BMPR1A. In some aspects, the decrease is from about 10% to about 100%, or alternatively at least about 15%, 20%, 25%, 30%, 35%, 40%, 45%, 50%, 60%, 70%, 80%, 90%, 95%, 98% or 99%. In one aspect, the decrease is a decrease of transcription, translation, or generally expression. In another aspect, the decrease is a decrease of protein activity.

A “substantially homogenous population” of cells refers to a population of cells that share the desired properties. A non-limiting example is a clonal population in which all, or a substantial portion, of the cells are produced from a common ancestor. A substantially homogenous population of cells, however, does not have to be clonal. For instance, all cells in a population can be induced to exhibit a desired property by, e.g., transfection or induction. A “desired property”, for instance, can be expression, up- or down-regulation of gene, attachment to a cell surface ligand, capable of hair generating, or undergoing propagation, without limitation. In one aspect, at least about 75%, 80%, 85%, 90%, 95%, 98% or 99% of the cells in the population share the common desired property.

As used herein, “alopecia” means loss of hair from the head or body of a mammalian subject, in particular a human. Alopecia is visible and medical diagnosis is also readily available. Alopecia can mean baldness, a term generally reserved for pattern alopecia or androgenic alopecia.

Certain other diseases and disorders are “associated with” or lead to alopecia. Non-limiting examples include alopecia mucinosa, androgenic alopecia diabetes, dissecting cellulitis, fungal infections (such as tinea capitis), hair treatments (chemicals in relaxers, hair straighteners), hereditary disorders, hormonal changes, hyperthyroidism and hypothyroidism, hypervitaminosis a, iron deficiency or malnutrition in general, lupus erythematosus, medications (side effects from drugs, including chemotherapy, anabolic steroids, and birth control pills), pseudopelade of brocq, radiation therapy, scalp infection, secondary syphilis, telogen effluvium, traction alopecia, trichotillomania, and tufted folliculitis.

“Administration”, as used herein, refers to the delivery of a medication, such as the agent of the invention, which inhibits or augments the BMP signaling, to an appropriate location of the subject, where a therapeutic effect is achieved. Non-limiting examples include oral dosing, intracutaneous injection, direct application to target area proximal areas on the skin, or applied on a patch. Various physical and/or mechanical technologies are available to permit the sustained or immediate topical or transdermal administration of macromolecules (such as, peptides). Such technologies include iontophoresis (see for example Kalia et al., Adv. Drug Del. Rev. 56:619-58, 2004) sonophoresis, needle-less injection, and/or microstructured arrays (sometimes called microneedles; one particular example is the Microstructured Transdermal System (MTS) commercially available from 3M) (see, e.g., Alain et al. (2002) J. Control. Release 81:113-119; Santi et al. (1997) Pharm. Res., 14(1):63-66; Sebastien et al. (1998) J. Pharm. Sci. 87(8):922-925). Methods of making and using arrays of solid microneedles that can be inserted into the skin for transdermal delivery of peptides (such as cyclic CRF antagonists) are provided in Martanto et al. (2004) Pharm. Res. 21:947-52, and Am. Inst. Chem. Eng. 51:1599-607 (2005). In some examples, the delivery system includes a combination of systems, such as microneedles made of biocompatible and biodegradable polymers (Park et al. (2005) J. Control. Release 104:51-66). Laser systems have also been developed to ablate the stratum corneum from the epidermal layer (Lee et al. (2002) J. Pharm. Sci. 91(7): 1613-1626). The laser-ablated regions offer lower resistance to drug (peptide) diffusion than non-ablated skin. In one aspect, administration is topical administration as defined herein.

“Topical administration” refers to delivery of a medication by application to the skin. Non-limiting examples of topical administration include any methods described under the definition of “administration” pertaining to delivery of a medication to the skin.

A penetration or permeation enhancer refers to a chemical composition or mechanical/electrical device that can increase the transdermal drug delivery efficiency. In one aspect, a penetration or permeation enhancer is soluble in the formulation and act to reduce the barrier properties of human skin. The list of potential skin permeation enhancers is long, but can be broken down into three general categories: lipid disrupting agents (LDAs), solubility enhancers, and surfactants. LDAs are typically fatty acid-like molecules proposed to fluidize lipids in the human skin membrane. Solubility enhancers act by increasing the maximum concentration of drug in the formulation, thus creating a larger concentration gradient for diffusion. Surfactants are amphiphilic molecules capable of interacting with the polar and lipid groups in the skin (see e.g. Francoeur et al., Potts, Russell 0. (1990) Pharm. Res. 7:621-7; U.S. Pat. No. 5,503,843).

A “composition” is intended to mean a combination of active agent, cell or population of cells and another compound or composition, inert (for example, a detectable agent or label or biocompatible scaffold) or active, such as a growth and/or differentiation factor.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG). These formulations may optionally comprise additional pharmaceutically acceptable ingredients such as diluents, stabilizers, and/or adjuvants. The pharmaceutically acceptable carrier facilitates immediate or controlled release of the active ingredient.

A “subject” of diagnosis or treatment is a cell or a mammal, including a human. Non-human animals subject to diagnosis or treatment include, for example, murine, such as rats, mice, canine, such as dogs, leporids, such as rabbits, livestock, sport animals, and pets.

An “effective amount” is an amount sufficient to effect beneficial or desired results. An effective amount can be administered in one or more administrations, applications or dosages.

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder, e.g., alopecia. As is understood by those skilled in the art, “treatment” can include systemic amelioration of the symptoms associated with the pathology and/or a delay in onset of symptoms such as hair loss.

Various proteins are also disclosed herein with their GenBank Accession Numbers for their human proteins and coding sequences. However, the proteins are not limited to human-derived proteins having the amino acid sequences represented by the disclosed GenBank Accession numbers, but may have an amino acid sequence derived from other animals, particularly, a warm-blooded animal (e.g., rat, guinea pig, mouse, chicken, rabbit, pig, sheep, cow, monkey, etc.).

As used herein, the term “Wnt7a”, or “wingless-type MMTV integration site family, member 7A” refers to a protein having an amino acid sequence substantially identical to any of the representative WNt7a sequences of GenBank Accession Nos. NP_{—}004616.2 (human), NP_{—}033553.2 (mouse) or NP_{—}001093943.1 (rat). Suitable cDNA encoding Wnt7a are provided at GenBank Accession Nos. NM_{—}004625.3 (human), NM_{—}009527.3 (mouse) or NM_{—}001100473.1 (rat).

As used herein, the term “biological activity of Wnt7a” refers to any biological activity associated with the full length native Wnt7a protein. In suitable embodiments, the Arih2 biological activity is equivalent to the activity of a protein having an amino acid sequence represented by GenBank Accession No. NP_{—}004616.2, NP_{—}033553.2 or NP_{—}001093943.1. Measurement of transcriptional activity can be performed using any known method, such as immunohistochemistry, reporter assay or RT-PCR.

As used herein, the term “Wnt7b”, or “wingless-type MMTV integration site family, member 7b” refers to a protein having an amino acid sequence substantially identical to any of the representative WNt7b sequences of GenBank Accession Nos. NP_{—}478679.1 (human), NP_{—}033554.3 (mouse) or NP_{—}001009695.1 (rat). Suitable cDNA encoding Wnt7b are provided at GenBank Accession Nos. NM_{—}058238.2 (human), NM_{—}009528.3 (mouse) or NM_{—}001009695.1 (rat).

As used herein, the term “biological activity of Wnt7b” refers to any biological activity associated with the full length native Wnt7b protein. In suitable embodiments, the Arih2 biological activity is equivalent to the activity of a protein having an amino acid sequence represented by GenBank Accession No. NP_{—}478679.1, NP_{—}033554.3 or NP_{—}001093943.1. Measurement of transcriptional activity can be performed using any known method, such as immunohistochemistry, reporter assay or RT-PCR.

As used herein, the term “Fzd10”, or “frizzled family receptor 10” refers to a protein having an amino acid sequence substantially identical to any of the representative Fzd10 sequences of GenBank Accession Nos. NP_{—}009128.1 (human) or NP_{—}780493.1 (mouse). Suitable cDNA encoding Fzd10 are provided at GenBank Accession Nos. NM_{—}007197.3 (human) or NM_{—}175284.3 (mouse).

As used herein, the term “biological activity of Fzd10” refers to any biological activity associated with the full length native Fzd10 protein. In suitable embodiments, the Arih2 biological activity is equivalent to the activity of a protein having an amino acid sequence represented by GenBank Accession No. NP_{—}009128.1 or NP_{—}780493.1. Measurement of transcriptional activity can be performed using any known method, such as immunohistochemistry, reporter assay or RT-PCR.

### DETAILED DESCRIPTION

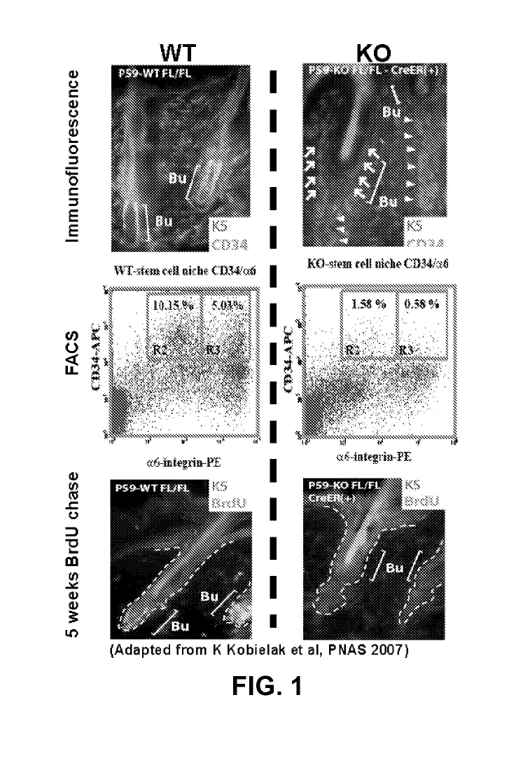

Although hair follicle stem cells are one of the best-characterized adult stem cell systems, it still is not known how BMP signaling integrates different regulators into a molecular network capable of cyclic activation of hfSCs. The main obstacle to address this important question in stem cell biology was due to the loss of expression of CD34, the only available marker for isolation of hfSCs upon BMP inhibition (FIG. 1).

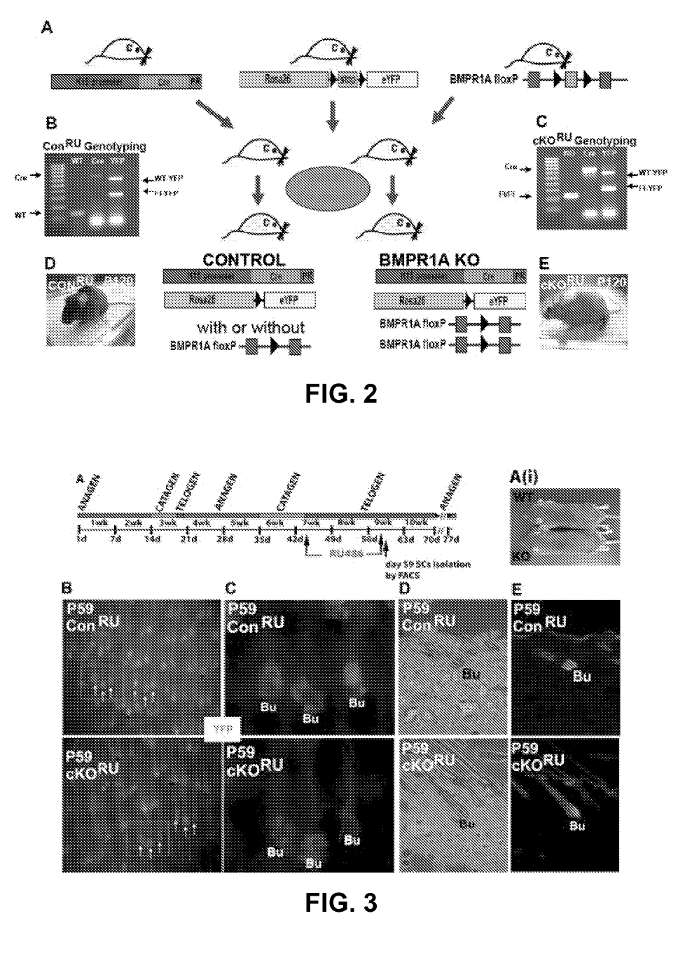

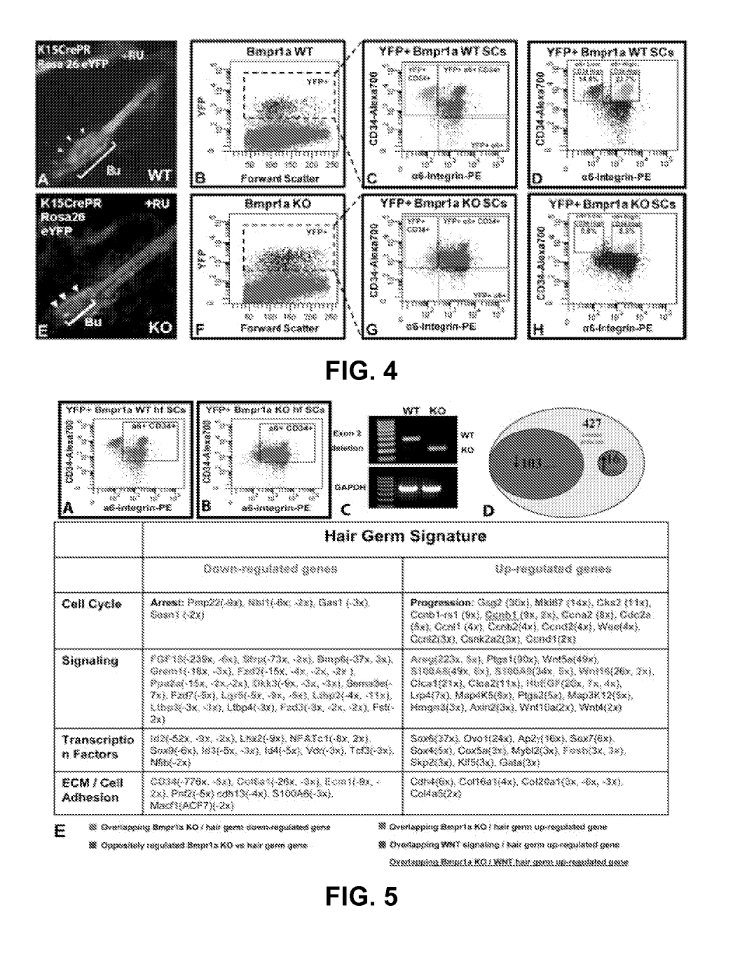

Therefore, the current challenge was to fill this gap and develop strategies which would allow one to isolate, characterize and culture hfSCs cells where BMP signaling was inactivated. To address this question the present disclosure employed, a Keratin 15 (K15)-driven genetic model to simultaneously label and specifically inactivate the BMP pathway within the hfSCs (FIG. 2). Using this approach for the first time the present inventors were able to isolate live hfSCs marked by eYFP that contained a BMPR1A knockout (KO) (FIG. 3). This was done by fluorescence activated cell sorting (FACS) (FIG. 4).

Subsequently, the present inventors were able to analyze the transcription profile of hfSCs just after inactivation of BMP signaling and before morphological changes occurred in hair follicle cycling. Thus, for the first time, the present inventors were able to look at very early changes in gene expression regulated by BMP signaling. This analysis reveals that after ablation of BMP signaling in the hf bulge, stem cells show down regulation of approximately 25% of common up regulated hfSCs signature genes (FIG. 5D).

At the same time, upon BMP inactivation hfSCs acquire molecular characteristics of the hair germ as the data show that 30% of genes overlapped with the previously characterized hair germ signature (FIG. 5E). Interestingly, it was found that hfSCs with suppressed BMP signaling show profound altered expression in the BMP pathway itself (known as an inhibitor of hair regeneration), Wnt pathway (known as an activator of hair regeneration), and Id (known as an inhibitor of differentiation) with undemonstrated function in hair regeneration.

These new findings illustrate a model where a constant competitive cycling between activator and inhibitor activities in hfSCs is critical to maintain hfSC homeostasis (FIG. 9). It is contemplated that BMP signaling holds hfSCs at a quiescent stage by interacting with several molecular pathways, including Wnts. Any event that can tilt the BMP-Wnt balance can lead to stochastic, yet temporal activation of some hfSCs (FIG. 9). Subsequently the release of hfSC self renewal can reset the balance back to quiescence within the bulge, thereby achieving a cyclic molecular network. Dermal papilla and subcutaneous adipose tissue in the vicinity of hfSCs work as signaling modulator centers to temporally either shorten or extend a period of cycling therefore balancing the molecular network inside hfSCs to promote either SC activation or inhibition.

In agreement to the model, it was observed the opposite regulation of hfSC homeostasis in the mice model were BMP signaling was constitutively activated. In these animals the morphological bulge position is intact and cells maintain quiescence but the biochemical markers of hfSCs (i.e., CD34 and K15-GFP) disappear; whereas the hair germ is converted into small non cycling cysts. In this animal model the present disclosure successfully reestablish hair follicle stem cells and cyclic regeneration by reactivating the molecular network.

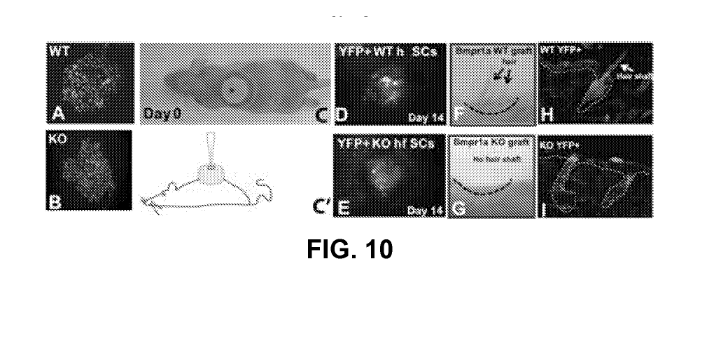

Hence, this provides a very powerful tool for the future understanding of hfSC regulation and formation. Surprisingly, even though 30% of the genes in these BMP inhibited hfSCs overlapped with the hair germ signature, these cells behaved differently than hair germ cells published previously. First the present inventors were able to maintain these cells in culture in vitro for a long time (>30 passages) (FIG. 10). Secondly, when the present inventors transplanted them back to the immune-compromised mice, they were able to regenerate epidermis, sebaceous glands and undifferentiated hair (without a hair shaft) (FIG. 10).

Additionally, Applicants also established and cultured the hfSCs from a double transgenic inducible system where BMP signaling was constitutively activated upon stimulation (FIG. 18). Thus, the present technology establishes unique in vitro systems to test different candidate genes regulated by BMP signaling that were discovered in the preliminary data in both loss and gain of function models.

Since, in both cases these cells maintain their stem cell characteristics during long term culture conditions, as well as when transplanted in vivo, this provides powerful tools to test different candidate genes involved in the BMP network and these allows to go back and test their function in vivo.

For all in vitro and then in vivo manipulations, a tet inducible improved lentiviral system was generated where the gene of interest (GOI) will be regulated tightly (without leakiness) by Doxycycline treatment and will be used universally to efficiently overexpress genes both in vitro and in vivo. Thus this allows to modify the original cells to either gain or lose gene function with the lentiviral vectors in vitro and vivo.

Additionally, this disclosure discloses new methods to visualize the spatio-temporal cyclic self-regulation of BMP signaling in hfSCs in vivo. This allows determination of how re-balancing BMP signaling levels in a BMP gain of function model can re-establish cyclic self-regulation of BMP signaling in hfSCs.

The present inventors also will investigate whether re-sensing BMP signaling in the BMP loss of function model, after quiescence of hfSCs has been disrupted, can re-activate the cyclic regulation of BMP signaling in vivo and re-establish hfSCs homeostasis.

Further, the K15-CrePR1/R26YFP or previous K15CrePR1 on Rosa26-loxP-stop-Lacz (R26lacZ) system will allow one to perform lineage tracing analysis in vivo to study the role of cell-autonomous and non cell-autonomous mechanisms of BMP signaling on the overall integrity of the hfSC pool and their homeostasis.

Overall, both the gain and loss of BMP function animal models are excellent and unique tools to address the questions of how BMP signaling integrates different regulators into a molecular network capable of cyclic activation of hfSCs. Additionally, the reversibility of the gain of function model gives us a unique opportunity to investigate how K15-GFP positive cells that are mispositioned within a hair-cyst can re-emerge in new hair follicle regeneration and how they contribute to new hfSCs repopulation and homeostasis.

These results indicate that there is a constant competitive cycling between activator and inhibitor activities in hfSCs population within the hair bulge microenvironment which is critical for maintenance of hfSCs homeostasis (FIG. 9). hfSCs constantly sum up surrounding activator and inhibitor activities and its functional states shift stochastically between activated, oscillating and quiescent states (FIG. 9).

When sufficiently tilted toward activators, those hfSCs are activated towards hair germ cells characteristic. When sufficiently tilted toward inhibitors, hfSCs are quiescent. When balanced, overall homeostasis of the hfSC population is maintained with a stochastic probability to undergo cyclic activation and quiescence (FIG. 9). A major source of activators is the dermal papilla which is pivotal in driving hair cycling. Extrinsic to the follicle, the dermal macro-environment can provide BMP to modulate this cycling process.

The corollary is that one should be able to visualize the activation of different percentages of hfSCs at different hair cycling stages. With gain and loss of function model systems that alter the level of BMP signaling in hfSCs, one can observe the consequence on the molecular network, coupled with changes in hair follicle activation or inhibition.

It is further contemplated that it is possible to re-establish hair follicle stem cells and cyclic regeneration by reactivating the molecular network, thus paving the road for future understanding of hair follicle stem cell formation.

Isolated Hair Follicle Stem Cell (hfSC) with Inhibited BMP Signaling

As disclosed herein, Applicants successfully isolated hfSC with inhibited BMP signaling. Thus, one embodiment of the present disclosure provides an isolated hair follicle stem cell (hfSC) that expresses keratin 15 (K15) and has decreased BMP signaling and populations of such cells, e.g., substantionally homogenous populations of such cells.

In one aspect, the hfSC has decreased biological activity of any member of BMP signaling genes such as, but not limited to BMP1, BMP2, BMP3, BMP4, BMP5, BMP6, BMP7, BMP8a, BMP8b, BMP10, BMP15 and BMPR1A. In a particular aspect, the hfSC has decreased biological activity of BMPR1A. In some aspects, the decrease activity is decreased expression. In some aspects, the decreased activity is no activity or no expression, such as a knock out gene.

In one aspect, the hfSC does not express CD34.

In one aspect, the isolated hfSC further comprises an exogenous reporter gene. The reporter gene is useful in monitoring the status of the hfSC and examples include the luciferase, the green fluorescent gene, the yellow fluorescent gene and alike.

In some aspects, the reporter gene is activated by a K15 promoter, which can be placed upstream of the reporter gene. In some aspects, the activation of the reporter gene is mediated by a pharmaceutical agent that is in contact with the hfSC.

In one aspect, the hfSC has increased expression of one or more of Wnt7a, Wnt7b, or Fzd10 and/or decreased expression of Dkk3 each as compared to a hfSC having normal BMP signaling.

Also provided, in one embodiment, is population of isolated hfSC of any of the above embodiments. In one aspect, the population is substantially homogenous. Further provided is a substantially homogenous population of cells differentiated from an isolated hfSC any of the above embodiments.

Further provided is a pharmaceutical composition comprising an isolated hfSC, a cell population of any of the above embodiments, and a pharmaceutically acceptable carrier.

In another embodiment, the present disclosure provides a method of treating alopecia in a mammalian subject, comprising implanting to the subject an isolated hair follicle stem cell (hfSC), a cell population, or a pharmaceutical composition of any of the above embodiments. Such methods are also useful in treating a scarred tissue, or any disease or condition characterized by a hair follicle cell unable to produce hair.

Also provided are methods of using these isolated hfSC to identify genes regulated by BMP signaling comprising comparing the activity or expression of a gene before and after inhibition of BMP signaling.

Isolated Hair Follicle Stem Cell (hfSC) Comprising Constitutively Active BMPR1A Gene

Inducible hfSC in which BMP signaling can be constitutively activated are also generated, providing a system for screening for agents suitable for activating hair germ cells.

Thus, in one embodiment, the present disclosure provides an isolated hair follicle stem cell (hfSC) comprising a constitutively active BMPR1A gene. Likewise, also provided are isolated hair follicle stem cells (hfSC) comprising a constitutively active BMP gene. Examples of BMP gene include BMP1, BMP2, BMP3, BMP4, BMP5, BMP6, BMP7, BMP8a, BMP8b, BMP10 and BMP15, without limitation.

In one aspect, the BMPR1A or BMP gene is regulated by a promoter inducible by a pharmaceutical agent that is in contact with the hfSC. In one aspect, the promoter is double Tg (dTg). In another aspect, the pharmaceutical agent is doxcycline.

In some aspects, the isolated hfSC further comprises a reporter gene regulated by a keratin 15 (K15) promoter.

Also provided, in one embodiment, is population of isolated hfSC of any of the above embodiments. In one aspect, the population is substantially homogenous. Further provided is a substantially homogenous population of cells differentiated from an isolated hfSC any of the above embodiments.

Further provided is a pharmaceutical composition comprising an isolated hfSC, a cell population of any of the above embodiments, and a pharmaceutically acceptable carrier.

Also provided is a transgenic non-human mammal comprising a hair follicle stem cell (hfSC) comprising a constitutively active BMPR1A or BMP gene. In some aspects, the hfSC further comprises a reporter gene regulated by a keratin 15 (K15) promoter.

### Drug Screen

The hfSC or transgenic animal having an inducibly constitutively activated BMP signaling can be useful for screening for agents suitable for inhibiting BMP signaling. In this respect, a reporter gene is particularly useful.

Thus, the present disclosure in one embodiment provides a method of identifying an agent suitable for inhibiting BMP signaling in a hair follicle, the method comprising contacting a candidate agent with an isolated hfSC or a genetically engineered non-human mammal having an inducibly constitutively activated BMP signaling, wherein expression of the reporter gene indicates that the candidate agent is suitable for inhibiting BMP signaling in a hair follicle.

Likewise, also provided is method of identifying an agent suitable for activating a hair follicle stem cell (hfSC), the method comprising contacting a candidate agent with an isolated hfSC or a genetically engineered non-human mammal having an inducibly constitutively activated BMP signaling, wherein expression of the reporter gene indicates that the candidate agent is suitable for activating a hfSC.

### Therapy

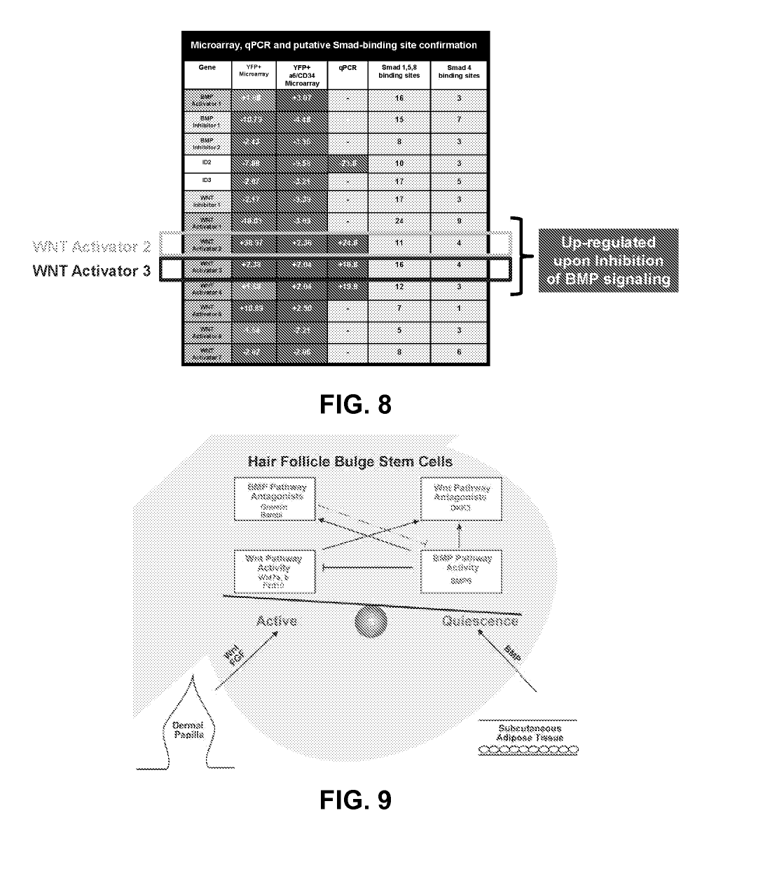

It is discovered that certain genes (Table 1, FIG. 8) are up- or down-regulated upon inhibition of BMP signaling. It is thus contemplated that by up- or down-regulating these genes in hfSC not having inhibited BMP signaling, BMP signaling in these hsSC can be disrupted, leading to activation of the hfSC. Further, it was discovered that, upon normalization of BMP signaling, new hair growth is reinitiated, demonstrating the effectiveness of promoting hair growth via BMP signaling normalization.

Therefore, in one embodiment, the present disclosure provides a method of activating a hair follicle stem cell (hfSC), comprising increasing the biological activity of one or more of Wnt7a, Wnt7b, or Fzd10 and/or decreasing the biological activity of Dkk3 in the cell.

In one aspect, the increasing of the biological activity of one or more of Wnt7a, Wnt7b, or Fzd10 comprises increasing of transcription of one or more of Wnt7a, Wnt7b, or Fzd10 in the cell.

In another aspect, the decreasing of the biological activity of Dkk3 comprises decreasing the transcription of Dkk3 in the cell.

In another aspect, the method further comprises contacting the hfSC with an agent that inhibits BMP signaling.

Methods of increasing or decreasing the biological activity or transcription of a gene are well known in the art and further described in the following section.

Also contemplated is a method of treating alopecia and related disorders in a mammalian subject, comprising implanting to the subject an activated a hair follicle stem cell (hfSC), wherein the hfSC was isolated from the subject and activated by a method of any of the above embodiments. Such methods are also useful in treating a scarred tissue, or any disease or condition characterized by a hair follicle cell unable to produce hair.

### Pharmaceutical Compositions and Therapies

The terms “nucleic acid”, “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown.

The following are non-limiting examples of polynucleotides: a gene or gene fragment (for example, a probe, primer, EST or SAGE tag), exons, introns, messenger RNA (mRNA), transfer RNA, small interference RNA (siRNA), double strand RNA (dsRNA), ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

Methods of decreasing the expression or activity of a gene include, without limitation, the use of the RNAi technology, the antibody technology or small molecule inhibitors.

“RNA interference” (RNAi) refers to sequence-specific or gene specific suppression of gene expression (protein synthesis) that is mediated by short interfering RNA (siRNA).

“Short interfering RNA” (siRNA) refers to double-stranded RNA molecules, generally, from about 10 to about 30 nucleotides long that are capable of mediating RNA interference (RNAi)), or 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, or 29 nucleotides in length. As used herein, the term siRNA includes short hairpin RNAs (shRNAs).

The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long. For example, the stem can be 10-30 nucleotides long, 12-28 nucleotides long, 15-25 nucleotides long, 19-23 nucleotides long, or 21-23 nucleotides long.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

Gene delivery vehicles also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells or cardiomyocytes. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

A “transgenic animal”, as used herein, refers to a non-human animal comprising an expression cassette, or a heterologous nucleic acid stably integrated into the animal genome, which expression cassette comprises a polynucleotide encoding a \ protein, including but not limited to Wnt7a, Wnt7b, or Fzzd10, under control of a skin-specific promoter, such as the keratin 14 promoter. The heterologous nucleic acid is introduced into the animal by genetic engineering techniques, such as by trangenic techniques known by those skilled in the art. In another aspect, the expression cassette comprises a polynucleotide encoding a BMP antagonist, such as noggin, chordin, gremlin, sclerostin and follistatin. More details of constructing the expression cassette and transgenic animal are described in Pilkus et al. (2004) Am. J. Pathol. 164:1099-114.

The term “expression cassette” or “transgenic gene construct” refers to a nucleic acid molecule, e.g., a vector, containing the subject gene, e.g., Wnt7a, Wnt7b, or Fzzd10, operably linked in a manner capable of expressing the gene in a host cell. The expression cassette or gene construct can be introduced into a non-human animal cell by nucleic acid-mediated gene transfer by methods known to those skilled in the art.

In certain aspects, the invention is related to an isolated or recombinant BMP protein, polypeptide BMP agonist or antagonist, examples of which are described herein as well as in Yanagita (2009) BioFactors 35(2):113-119. Yanagita (2009) supra., reports BMP antagonists and agonists known in the art. Agonists include repulsive guidance molecule (RGMA), DRAGON (RGMB), hemojuvelin, kielin/chordin-like protein (KCP), and Crossveinless 2 (Cv2). Antagonists include chordin, noggin, the eight-membered rings Dan family, the nine-membered ring Tsg family and Crim1. Also encompassed by this invention are polypeptides having at least 80% sequence identify, or alternatively 85% sequence identify, or alternatively 90% sequence identity, or alternatively 95% sequence identify, to these polypeptide agonists and antagonists.

Polypeptides of the invention can be prepared by expressing polynucleotides encoding the polypeptide sequences of this invention in an appropriate host cell. This can be accomplished by methods of recombinant DNA technology known to those skilled in the art. Accordingly, this invention also provides methods for recombinantly producing the polypeptides of this invention in a eukaryotic or prokaryotic host cells. The proteins and polypeptides of this invention also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

It is known to those skilled in the art that modifications can be made to any peptide to provide it with altered properties. Polypeptides of the invention can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the invention as retro-inverso peptides. In addition, the present invention envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present invention provides a method to produce a constrained peptide that incorporates random sequences at all other positions.

The following non-classical amino acids may be incorporated in the peptides of the invention in order to introduce particular conformational motifs: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai and Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013, issued Aug. 8, 1995 to Kahn.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are know to one of skill in the art. Non-limiting examples include empirical substitution models as described by Dahoff et al. (1978) In Atlas of Protein Sequence and Structure Vol. 5 suppl. 2 (ed. M. O. Dayhoff), pp. 345-352. National Biomedical Research Foundation, Washington D.C.; PAM matrices including Dayhoff matrices (Dahoff et al. (1978), supra, or JTT matrices as described by Jones et al. (1992) Comput. Appl. Biosci. 8:275-282 and Gonnet et al. (1992) Science 256:1443-1145; the empirical model described by Adach and Hasegawa (1996) J. Mol. Evol. 42:459-468; the block substitution matrices (BLOSUM) as described by Henikoff and Henikoff (1992) Proc. Natl. Acad. Sci. USA 89:10915-10919; Poisson models as described by Nei (1987) Molecular Evolutionary Genetics. Columbia University Press, New York; and the Maximum Likelihood (ML) Method as described by Müller et al. (2002) Mol. Biol. Evol. 19:8-13.

The polypeptides and polypeptide complexes of the invention can be used in a variety of formulations, which may vary depending on the intended use. For example, one or more can be covalently or non-covalently linked (complexed) to various other molecules, the nature of which may vary depending on the particular purpose. For example, a peptide of the invention can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome, see U.S. Pat. No. 5,837,249. A peptide of the invention can be complexed covalently or non-covalently with a solid support, a variety of which are known in the art and described herein. An antigenic peptide epitope of the invention can be associated with an antigen-presenting matrix such as an MHC complex with or without co-stimulatory molecules.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable cross-linking agents are cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound may be used. Also included are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homo-bifunctional cross-linking agents include the bifunctional N-hydroxysuccinimide esters dithiobis(succinimidylpropionate), disuccinimidyl suberate, and disuccinimidyl tartrate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio) propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of common hetero-bifunctional cross-linking agents that may be used to effect the conjugation of proteins to peptides include, but are not limited to, SMCC (succinimidyl-4-(N-maleimidomethyl)cyclohexane-1-carboxylate), MBS (m-maleimidobenzoyl-N-hydroxysuccinimide ester), SIAB (N-succinimidyl(4-iodoacteyl)aminobenzoate), SMPB (succinimidyl-4-(p-maleimidophenyl)butyrate), GMBS (N-(γ-maleimidobutyryloxy)succinimide ester), MPBH (4-(4-N-maleimidopohenyl) butyric acid hydrazide), M2C2H (4-(N-maleimidomethyl)cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Cross-linking may be accomplished by coupling a carbonyl group to an amine group or to a hydrazide group by reductive amination.

Peptides of the invention also may be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein. Biotinylated peptides then can be incubated with avidin or streptavidin to create large complexes. The molecular mass of such polymers can be regulated through careful control of the molar ratio of biotinylated peptide to avidin or streptavidin.

Also provided by this application are the peptides and polypeptides described herein conjugated to a label, e.g., a fluorescent or bioluminescent label, for use in the diagnostic methods. For example, detectably labeled peptides and polypeptides can be bound to a column and used for the detection and purification of antibodies. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in Haugland, Richard P. (1996) Molecular Probes Handbook.

The polypeptides of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete Adjuvant, Freund's Incomplete Adjuvant and mineral salts.

In one aspect, the invention provides compositions used in the methods. In some embodiments, the compositions are small molecules that enhance or inhibit BMP signaling. In some embodiments, the compositions are polynucleotides that encode BMP proteins or BMP target proteins, such as Wnt7a, Wnt7b, or Fzzd10. In some embodiments, the compositions are isolated or recombinant proteins. In some aspect, the composition further comprises a pharmaceutically acceptable carrier. In another aspect, the compositions contain carriers that modulate (controlled release) the release of the active agent when administered to a subject in need thereof.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The compositions of this invention are formulated for pharmaceutical administration to a mammal, preferably a human being. Such pharmaceutical compositions of the invention may be administered in a variety of ways, preferably topically or intradermally.

Pharmaceutically acceptable excipients and carriers and dosage forms are generally known to those skilled in the art and are included in the invention. It should be understood that a specific dosage and treatment regimen for any particular patient will depend upon a variety of factors, including the activity of the specific antidote employed, the age, body weight, general health, sex and diet, renal and hepatic function of the patient, and the time of administration, rate of excretion, drug combination, judgment of the treating physician or veterinarian and severity of the particular disease being treated.

For prophylactic administration, the compound can be administered to a patient at risk of developing one of the previously described conditions. For example, prophylactic administration can be applied to avoid the onset of symptoms in a patient diagnosed with the underlying disorder such as alopecia or a genetic predisposition to alocpecia.

The amount of compound administered will depend upon a variety of factors, including, for example, the particular indication being treated, the mode of administration, whether the desired benefit is prophylactic or therapeutic, the severity of the indication being treated and the age and weight of the patient, and the bioavailability of the particular active compound. Determination of an effective dosage is well within the capabilities of those skilled in the art.

Effective dosages can be estimated initially from in vitro assays. For example, an initial dosage for use in animals can be formulated to achieve a local (topical) or circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

Polypeptides may also be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein.

The polypeptides also can be combined with various liquid phase carriers, such as sterile or aqueous solutions, pharmaceutically acceptable carriers, suspensions and emulsions for immediate or controlled release. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete Adjuvant, Freund's Incomplete Adjuvant and mineral salts.

### Administration

Compositions described above, such as polynucleotides or polypeptides of Wnt7a, Wnt7b, or Fzd10, can be administered to the subject in need of. In one aspect, the composition is directly delivered into skin. In another aspect, the composition is delivered during telogen phase of the hair follicle which can be determined by one skilled in the art and briefly described herein. In another aspect, delivery can be made via microneedles. Microneedles allow penetrating stratum corneum—the outer layer of epidermis, responsible for the most of skin's barrier properties. Since microneedles do not reach into deeper skin layer, they do not cause painful sensations.

The proteins can be delivered intracutaneously via single glass microneedles. Delivery of BMP proteins during competent telogen phase rendered treated skin refractory and prevented hair regeneration. For more standardized and simplified intracutaneous delivery hollow microneedle arrays can be used. Microneedle arrays contain hundreds of small individual microneedles evenly spaced apart on a platform. Microneedle array can also be connected to protein reservoir and injection mechanism. Such delivery apparatus can be realized in form of disposable injection syringe. Alternative delivery platform can be based on principle of micro-fluidics. Microneedle/micro-fluidics device will provide slow intradermal delivery of compound at a constant rate over prolonged period of time. Such delivery platform can be realized in form of skin patch that can be attached over treatment area and worn without inconvenience for the patient.

Micro-needle arrays can be combined with syringe-like injection device to achieve simple protein delivery. Such delivery system can be realized in form of dermal patch, similar to ionophoretic insulin dermal patch.

Expression vectors, such as those expressing BMP ligands or antagonists, or naked cDNA for these genes can be delivered into skin using established intracutaneous gene delivery techniques, such as technique of electorporation or with the help of “gene gun”. In order to express the proteins described herein, delivery of nucleic acid sequences encoding the gene of interest can be delivered by several techniques as described herein. A polynucleotide can be delivered to a cell or tissue using a gene delivery vehicle. Gene delivery vehicles may also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens. Cell surface antigens characteristic to epidermis or hair follicle specific cell types should be used. Alternatively, antigens characteristic to stem cells should be used to target gene delivery into stem cells (such as hair follicle stem cells). In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

In one aspect, the composition further comprises a penetration enhancer or a carrier suitable for controlled release.

In some embodiments, compositions of the invention can be delivered into the skin by injection with a carrier for long term release and effect. In one aspect, beads are used as a protein reservoir. In another aspect, the composition further comprises a biocompatible and/or dissolvable carrier. Non-limiting examples of biocompatible and/or dissolvable carriers include injectable collagen matrix, dissolvable hydrogel and injectable biocompatible and dissolvable polymers.

In one aspect, the composition of this invention can be used to treat a condition in a mammalian subject in need of. In some embodiments, the condition comprises hair loss or insufficient hair growth. In one aspect, the condition is alopecia. In one aspect, the invention provides methods to prevent alopecia. In one aspect, the invention provides methods to inhibit BMP signaling in the skin.

In another aspect, the composition can be co-administrated, or administered prior to or after administration of a second agent that enhances hair growth. In one aspect, the second agent is minoxidil, a treatment for alopecia, commercially available as Rogaine or Regaine. In some embodiments, a combination of slow release excipients having two different rates of release where the composition of the invention is released over the course of a few hours, a day or more, followed by several days of release of the second agent. In another aspect, time release encapsulation comprising the compositions of the invention can be included in shampoo for convenient administration.

In one aspect, the invention provides methods to enhance hair growth or to treat alopecia thereby hair is formed on bald skin. In another aspect, the invention provides methods to enhance hair growth or to treat alopecia thereby the density of hair is increased or speed of hair growth is increased. In some embodiments the method further comprises laser ablating the tissue prior to administration of the agents. In some embodiments, the method further comprises administration of penetration enhancer prior to or concomitantly with administration of the agents.

The agents and compositions of the present invention can be used in the manufacture of medicaments and for the treatment of humans and other animals by administration in accordance with conventional procedures, such as an active ingredient in pharmaceutical compositions.

### Kits

An aspect of the invention provides a kit for promoting hair growth comprising an effective amount of an agent in a pharmaceutically acceptable carrier and instructions for use in promoting hair growth. Additional agents can be co-formulated or delivered concomitantly or sequentially with the above noted agents, e.g., minoxidil and provided in the kits. The formulations can be for immediate or controlled release of the active ingredients.

In some embodiments, the pharmaceutically acceptable carrier in the kits is suitable for topical administration of the agent. Additional agents can be co-formulated or delivered concomitantly or sequentially with the above noted agents, e.g., minoxidil. The formulations can be for immediate or controlled release of the active ingredients.

In some embodiments, the pharmaceutically acceptable carrier further comprises a penetration or permeation enhancer.

Also provided are kits for administration of the compounds for treatment of disorders as described herein. Kits may further comprise suitable packaging and/or instructions for use of the compound. Kits may also comprise a means for the delivery of the at least one agent and instructions for administration. Alternatively, the kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch, or inhalant.

The kits may include other therapeutic compounds for use in conjunction with the compounds described herein. These compounds can be provided in a separate form or mixed with the compounds of the present invention.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the invention, kits for treating an individual who suffers from or is susceptible to the conditions described herein are provided, comprising a container comprising a dosage amount of a composition, as disclosed herein, and instructions for use. The container can be any of those known in the art and appropriate for storage and delivery of oral, intravenous, topical, rectal, urethral, or inhaled formulations.

Kits may also be provided that contain sufficient dosages of the effective composition or compound to provide effective treatment for an individual for an extended period, such as a week, 2 weeks, 3, weeks, 4 weeks, 6 weeks, or 8 weeks or more.

The following examples are provide to illustrate select embodiments of the invention as disclosed and claimed herein.

### EXPERIMENTAL EXAMPLES

### Example 1

This example demonstrates the isolation and characterization, for the first time, of hair follicle stem cells (hfSCs) that have inactivated BMP signaling.

It is not known how BMP signaling integrates different regulators into a molecular network capable of cyclic activation of hfSCs. The main obstacle to address this important question in stem cell biology was the loss of expression of CD34, the only available marker for isolation of hfSCs upon BMP inhibition (FIG. 1).

Labeling Hair Follicle Stem Cells In Vivo after BMP Signaling Inhibition

It is discovered herein that in BMPR1A inducible conditional knockout mice (BMPR1A floxed mice) keratin 15 (K15) is still expressed in hfSCs. BMPR1A floxed mice were generated with specific inactivation of BMP signaling in hfSCs using a keratin 15 promoter (K15) driven recombinase (Cre) conjugated with a truncated progesterone receptor (PR), (K15-CrePR) (FIG. 2).

To simultaneously label in vivo hfSCs these mice were crossed with a Cre-dependent YFP (Yellow Fluorescent Protein) reporter knocked into the ubiquitously expressed Rosa26 locus (FIG. 2). After topical treatment with RU486 (RU) of the K15-CrePR/BMPR1A(fl/fl)/ROSA-YFP mice, any hfSCs that had expressed K15-CrePR now express YFP irreversibly. Offspring from matings of K15CrePR^{RU}/Bmpr1a(fl/+)/YFP(fl/+) mice yielded litters of the expected numbers, genotype and Mendelian ratios (FIG. 2).

RU was used to induce Cre-dependent recombination in adult mice at the time hair follicles were in the second postnatal extended and synchronized telogen (FIG. 3). Prior to RU treatment, K15CrePR^{RU}/Bmpr1a(fl/fl)/YFP(fl/+) or K15CrePR^{RU}/Bmpr1a(fl/fl)/YFP(fl/fl) (cKO) were indistinguishable from control (CON) animals which included: K15CrePR^{RU}/Bmpr1a(fl/+)/YFP(fl/+), K15CrePR^{RU}/Bmpr1a(fl/+)/YFP(fl/fl), K15CrePR^{RU}/Bmpr1a(+/+)/YFP(fl/+) and K15CrePR^{RU}/Bmpr1a(+/+)/YFP(fl/fl).

As expected at P120 after ablation of BMPR1A, an identical, very strong phenotype with visible hair loss was observed as previously reported for K14-CreER (Cre conjugated with a truncated form of estrogen receptor-ER) in this cKO^{RU} mice (FIG. 2), which confirmed high efficiency of recombination in vivo.

YFP Specific Activation of the hfSCs in BMPR1A Inducible Conditional Knockout Mouse.

At postnatal d43, during synchronized extended second telogen, mice were shaved and topically treated with RU for 16 d (FIG. 3A and Ai). Upon experimentally testing, 16 days of treatment was the optimal time to efficiently activate YFP expression specifically in hfSCs (FIGS. 3B, C and E). At postnatal d59, hair follicles (HFs) both cKO^{RU} and CON^{RU} HFs still exhibited a telogen morphology (FIGS. 3D and E).

Isolation of YFP Positive hfSCs after BMP Signaling Inhibition

A single cell suspension was then made from the whole back skin from either cKO^{RU} or CON^{RU} at P59 before the morphological changes occur between the WT and KO (FIGS. 4A and E). Using this approach the present inventors were able to isolate hfSCs marked by YFP from control and Bmpr1a KO by FACS (FIGS. 4B and F). Approximately 1-2% of the total cell population isolated from WT or KO back skin was YFP positive (FIGS. 4B and F, respectively). Then, these YFP positive populations were fractionated from WT and KO hfSCs (by employing α6-integrin and CD34 antibodies staining) into three distinct subpopulations: YFP+ α6+; YFP+ CD34+ (suprabasal hair follicle stem cells) and YFP+α 6+ CD34+ (basal hair follicle stem cells) (FIGS. 4C and G, respectively).

Although morphologically the hair follicles remained in the telogen phase of the hair cycle, it was confirmed that upon Bmp pathway inactivation, high expression of the CD34 marker in hfSCs was already decreased in Bmpr1a KO YFP+ cells (FIG. 4H), when compared with the same Bmpr1a WT YFP+ CD34 high fractions (FIG. 4D). That finding was expected.

Therefore, by virtue of the discovery of the K15 marker, for the first time, the present inventors were able to FACS isolate live YFP positive stem cells from hair follicles where BMP signaling was inactivated (FIGS. 4B and F).

### Example 2

With the hfSC isolated from Example 1, this example shows that the present inventors were able to examine molecular characteristics of these hair germ cells.

YFP Positive Bmpr1a KO hfSCs Acquire a Molecular Characteristic Towards that of the Hair Germ

To characterize target genes relevant for BMP signaling total RNAs from BMPR1A WT and KO basal hair follicle stem cells populations (b-hfSCs) YFP+α6+ CD34+ were used to perform microarray analysis (FIGS. 5A and B, respectively). High efficiency BMPR1A inactivation in KO hfSC populations was confirmed compared to WT by RT-PCR (FIG. 5C).

Patterns of expression, resulted from microarray analysis, revealed the down-regulation of approximately 25% (103 genes out of 427 probes) of commonly up-regulated hfSCs signature genes after inhibition of BMP signaling in hfSCs, (FIG. 5D).

Recently, Greco et al., characterized signature genes of hair germs (HG), a small cluster of cells appearing “periodically” during the telogen phase of the hair follicle cycle, between the hair follicle stem cells and dermal papilla (DP) (Greco et al., Cell Stem Cell 4(2):155-69 (2009)). It has been demonstrated that HG cells are derived from hfSCs (SCs) but become responsive quicker to DP-promoting signals in the new anagen phase of the hair cycle. The data in this example showed that after inactivation of BMP signaling, hfSCs acquired molecular characteristics towards that of the hair germ, since 30% of the genes overlapped with the previously characterized hair germ signature (FIG. 5E).

Changes in Expression of Wnt and BMP Signaling Pathway Genes in hfSCs after BMP Inhibition

Although it is demonstrated here that the inhibition of BMP signaling caused β-catenin stabilization and subsequent activation of the canonical WNT pathway, it can still not be ruled out if this mechanism is Wnt ligand-receptor dependant or independent.

Several genes in both pathways were found to be significantly either up or down regulated in BMPR1A KO hfSCs (Table 1 and FIG. 8). To check how consistent these gene changes are between BMPR1A WT and KO the present inventors performed and compared it with another microarray using the whole fractions of YFP positive hfSCs from independent biological samples (isolation as shown on FIGS. 4B and F).

TABLE 1
Molecular changes in genes of Wnt and BMP signaling
pathways in hfSCs after BMP inhibition

| | YFP+ | YFP+ a6+ | Name used |
|---|---|---|---|
| | only | CD34+ | in FIG. 8 |
| Bmp6 | 1.68 | 3.07 | BMP activator 1 |
| Bambi | −10.79 | −4.48 | BMP inhibitor 1 |
| Grem1 | −2.43 | −3.35 | BMP inhibitor 2 |
| Id2 | −7.69 | −9.58 | ID2 |
| Id3 | −2.07 | −3.21 | ID3 |
| Dkk3 | −2.17 | −3.39 | WNT inhibitor 1 |
| Wnt16 | 1.68 | 2.04 | WNT activator 4 |
| Wnt5b | −16.03 | −3.03 | WNT activator 1 |
| Wnt7a | 38.97 | 2.36 | WNT activator 2 |
| Wnt7b | 2.38 | 2.04 | WNT activator 3 |
| Fzd2 | −1.54 | −2.23 | WNT activator 6 |
| Fzd3 | −2.02 | −2.06 | WNT activator 7 |
| Fzd10 | 10.89 | 2.50 | WNT activator 5 |

The isolation of YFP positive cells for microarray comparison between WT and KO was performed at the same time point p59 during hair cycle (after the same regime of RU treatment at starting point at P43 for 16 days) (FIGS. 4B and F). The changes observed in the whole YFP positive fraction were consistent with patterns of gene expression of the YFP+α6+ CD34+ subpopulation (Table 1). These new findings suggest a model where a constant competitive cycling between activator and inhibitor in hfSCs is critical for maintenance of hfSC homeostasis (FIG. 9).

These data therefore show that BMP signaling holds hfSCs in a quiescent state by interacting with several molecular pathways, including Wnts. The data also show that any event that can tilt the BMP-Wnt balance can lead to stochastic yet temporal activation of some hfSCs (FIG. 9). Subsequently the release of hfSC self-renewal can reset the balance back to quiescence within the bulge, therefore achieving a cyclic molecular network. Dermal papilla and subcutaneous adipose tissue in the vicinity of hfSCs work as signaling modulator centers to temporally shorten or extend a period of cycling; thereby balancing the molecular network inside hfSCs to promote SC activation or inhibition (FIG. 9).

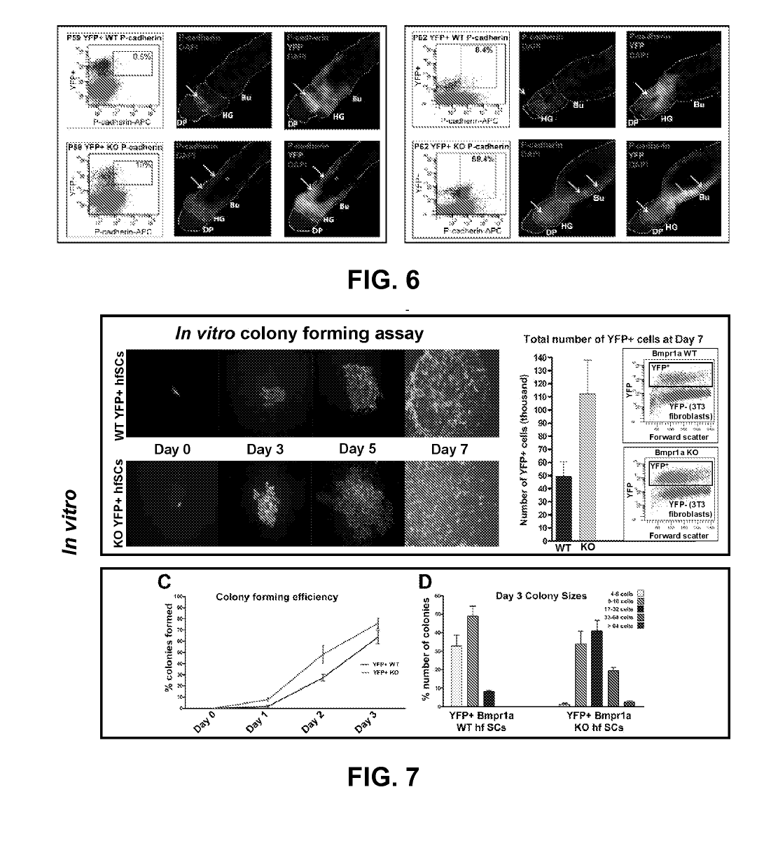

Further, as shown in FIG. 6, the hair germ marker, P-cadherin, is expanded in activated KO hf SCs. The hair germ (hg) arises from the bulge (bu) at the end of catagen and is found at hair follicle base in contact with the dermal papillae (dp). At the end of telogen, the hair germ is activated by the dp before the bulge SCs. In vitro, hair germ cells proliferate quickly but exhaust their proliferative potential. HG prevents depletion of the hfSCs and enables rapid initial hair growth in vivo. Also, Bmpr1a KO YFP+ hfSCs develop hair germ characteristics in vivo but maintain SC-like potential in vitro (FIG. 7). Like hg cells, Bmpr1a KO hfSCs proliferate faster in vitro but unlike hg cells, both YFP+ WT and KO hfSCs can be passaged many times (>P20) in vitro.

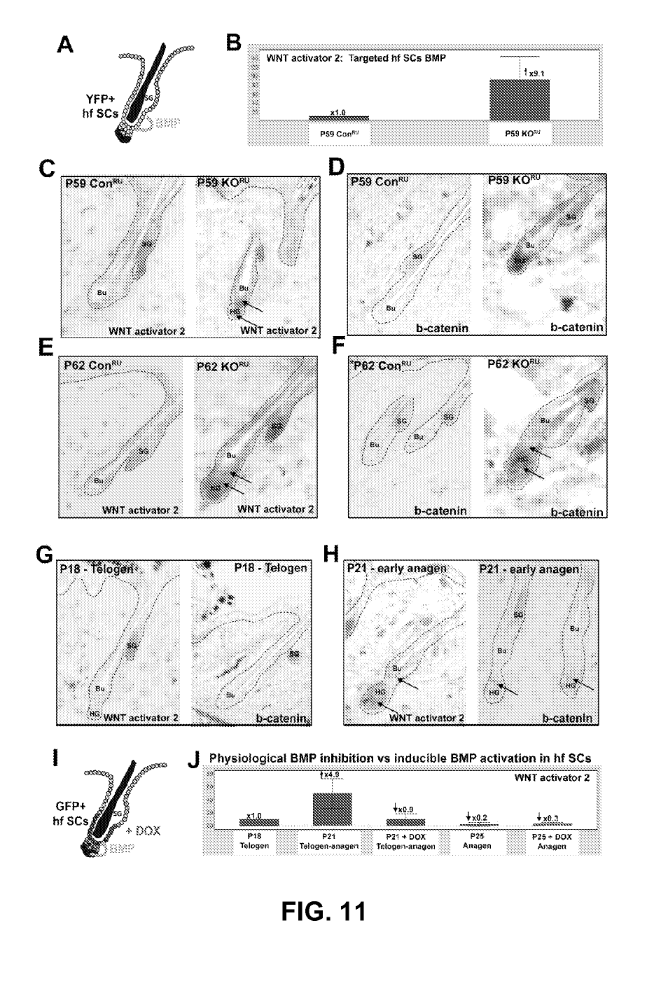

Wnt activators 2 and 3 (Wnt7a and Wnt7b respectively) are upregulated upon inhibition of BMP signaling (FIG. 8). BMP inactivation in hfSCs results in overexpression of WNT activator 2 in the bulge and hair germ and nuclear beta-catenin stabilization (FIG. 11). Physiological WNT activator 2 expression is observed in the bulge and hair germ during telogen to anagen transition and Beta-catenin stabilization. BMP activation, on the other hand, results in inhibition of WNT activator 2 expression in hf SCs (FIG. 11).

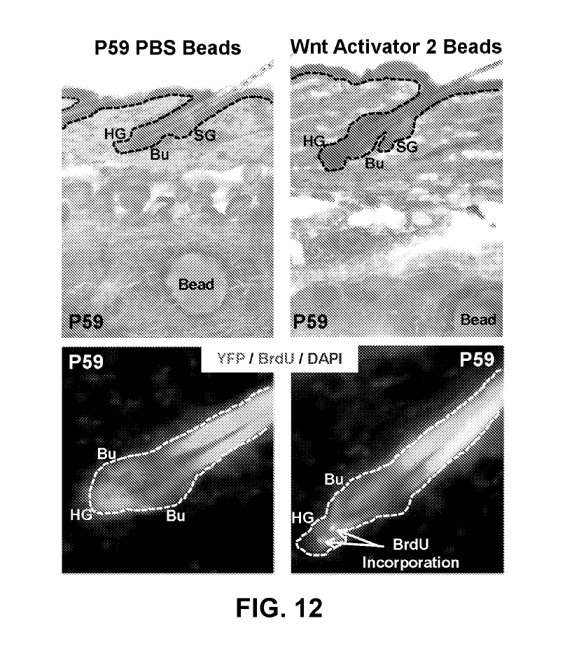

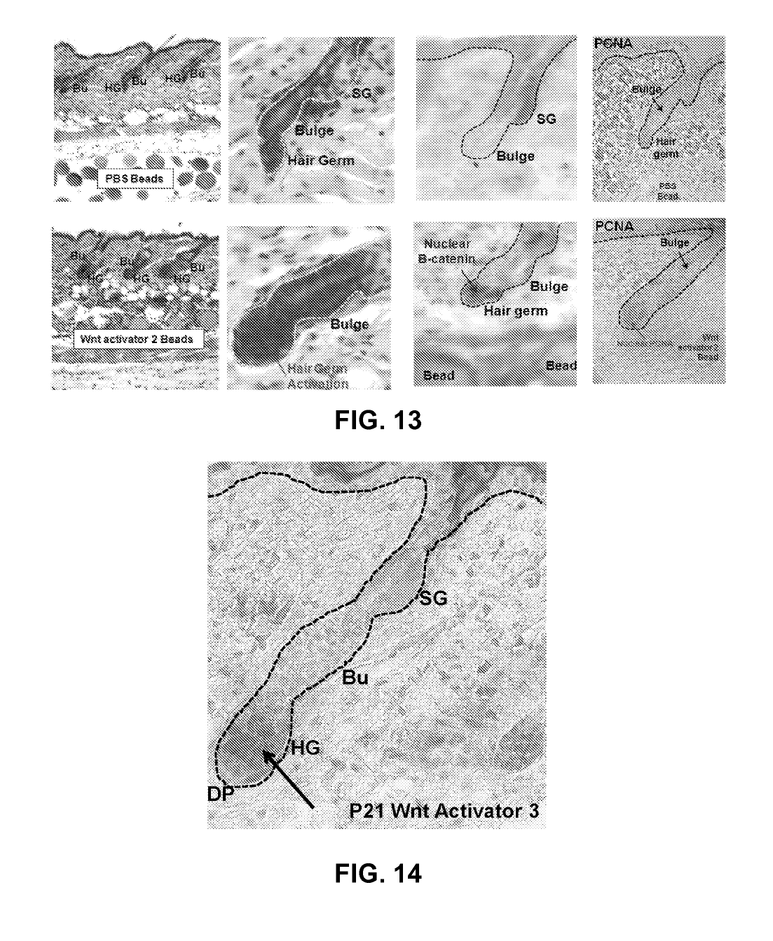

To further investigate the effect of WNT activator 2 in hair germ activation, mice received subcutaneous injection of WNT activator 2 recombinant protein. Such introduction of WNT activator 2 induced precocious hair germ activation (FIG. 12) and promoted precocious telogen to anagen transition (FIG. 13).

Likewise, Wnt Activator 3 is expressed physiologically during the telogen-anagen transition (FIG. 14). The data demonstrate that BMP signaling repress Wnt activator 3, and thus also show that inhibition of BMP signaling (seen during physiological telogen-anagen transition) promote Wnt Activator 3 expression. The question is then whether Wnt Activator 3 inhibition affects hair cycling in vivo.

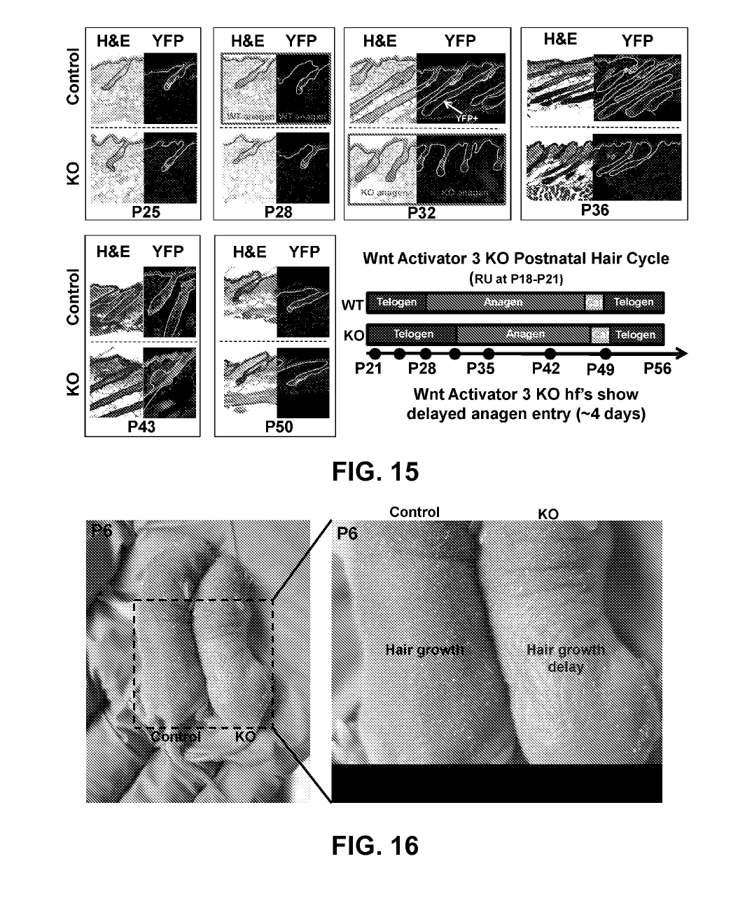

K15-driven, hf SCs Wnt Activator 3 deletion assay then showed that Wnt activator 3 inactivation delayed hair cycling (FIG. 15). During hair follicle morphogenesis, Wnt Activator 3 deletion delayed but did not prevent hair growth (FIG. 16).

### Example 3

This example investigates the self-regulation of BMP signaling in hfSCs.

On the basis of Examples 1 and 2 (Table 1 and FIG. 8) the model illustrated in FIG. 9 is contemplated, where constant competitive cyclic changes in BMP signaling activators and inhibitors happen inside hfSCs. The data show that BMP inhibition in hfSCs lead to a cell-autonomous secretion of BMP6 and suppression of the BMP antagonists Gremlin and Bambi (Table 1 and FIG. 9). Thus, this model suggests that the initial consequences of BMP inactivation will be temporal activation of hfSCs in a cell-autonomous manner in competent telogen hair follicles. Then, activated hfSCs will gradually start to express BMP6 and inhibit expression of Gremlin and Bambi.

These negative feedback loops will first release inhibited BMP signaling and eventually swing toward BMP activation leading to hfSCs quiescence. The feedback loop will then reverse direction after BMP reaches full activation in hfSCs, resulting in progressive activation of their own inhibitors (FIG. 9), completing the cycle. Thus, this example tests if the proposed cyclic regulation of activators (BMP6) or inhibitors (Bambi, Grem1) is directly regulated by BMP canonical signaling in hfSCs.

BMP6 and Gremlin were reported to be upregulated in hfSCs but their regulation or function has not been explored in vivo. Bambi function has not been studied in hfSCs yet, however recently Bambi was described as one of the genes negatively regulated by micro-RNA-31, which controls hair cycle-associated changes in vitro and in vivo. One explanation why these genes have been just sporadically reported so far is that all previous methods focus on isolation of hfSCs either in telogen or during the telogen to anagen transition. This approach mostly focused on relatively quiescent hfSCs as demonstrated recently. Even the early stage transition between telogen to anagen activates only a few cells in the hfSCS but predominantly in the hair germ.

Therefore, this example synchronizes the hfSCs in the bulge towards activation after BMP inhibition, provides a unique opportunity to look at these changes at a very early time point with higher resolution.

Additionally it was reported that BMP-2 caused a time- and dose-dependent increase in gremlin. However, a similar dependence relationship has not been reported between Bambi and BMP. Additionally this example tests known effectors of BMP signaling: id2, id3, whose expression responded in BMPR1A KO mice (FIG. 5E and Table 1) and was confirmed by Q-PCR. Just recently, BMP responsive elements in the id2 gene have been described. Additionally, id2, id3 showed dynamic changes in expression in vibrissae follicle development. Even though id genes have been proposed to be direct targets of BMP signaling, their role in hfSCs regulation and hair follicle cycling is not known.

To address the questions this example developed tools for both in vitro and vivo studies. The data showed even though YFP positive Bmpr1a KO hfSCs acquire molecular characteristic towards that of the hair germ, they behaved differently than the hair germ population, namely YFP labeled Bmpr1a KO hair follicle stem cells maintain characteristics of stem cells when cultured in vitro (FIG. 10B) and after transplantation in vivo (FIG. 10D-I).

Thus for the first time this example successfully isolated and culture hfSCs without BMPR1A (FIG. 10B), additionally this example confirmed their stem cell multipotency in vivo using chamber graft transplantation (FIG. 10D-I). This is a very useful system to test the BMP candidate genes both in vitro and in vivo.

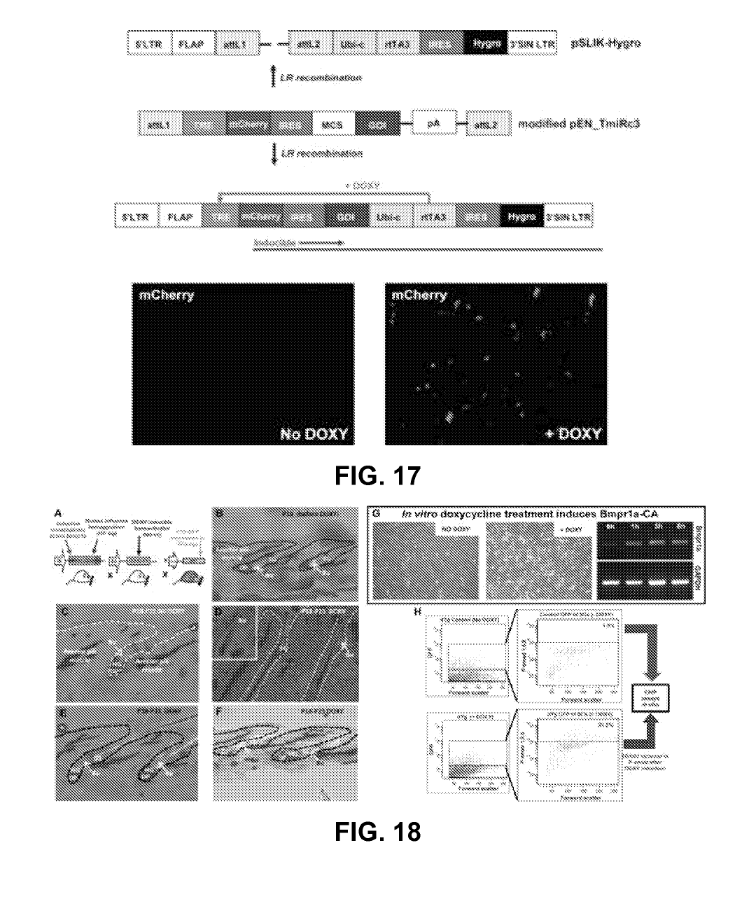

To do so, this example reconstructed a doxycycline-inducible lentiviral expression system with mCherry (FIG. 17).

Promoter regions of BMP6, Gremlin and Bambi are studied along with recently published constructs for id2 and id3 promoters as controls to find out active Smad binding elements (SBEs) within these target gene promoters. Briefly, this example clones the different regions of these promoters (up to 5 kb upstream from transcription's start) in to the pGL2-Promoter or pGL3-Promoter (Promega) cassette which contains the SV40 promoter upstream of the firefly luciferase gene (as a control of transfection efficiency this example will use the Renilla luciferase cassette from Promega). Then this example tests responsiveness to BMP activation or repression of these different promoter constructs in the BMP signaling loss (FIGS. 10A and B) and gain of function cell lines (FIG. 18G) after in vitro transfection.

Computer predictions (Biobase, BKL TRANSFAC and Genomatrix promoter analyses software) are used to analyze SMAD binding sites within the promoter regions of each selected BMP signaling target gene and confirmed the existence of several putative binding sites. This example then validates these putative SBE sites by performing site mutagenesis and then conducts in vivo chromatin immunoprecipitation (ChIP) assays, using an anti-P-Smad Ab (Cell Signaling) with the BMP6, Gremlin and Bambi promoters as well as id2 and id3 promoters as controls.

Investigation of the Biological Function and Mechanism of BMP Activator (Bmp6), Inhibitors (Bambi, Grem1) and Effectors (Id2, Id3) Upon Regulation of hfSCs.

Examples 1 and 2 demonstrated the successfully cultured hfSCs without BMPR1A (FIG. 10B), and used them in vivo in chamber graft transplantation (FIG. 10D-I). Thus it provides an attractive and powerful system to test the BMP candidate genes in vivo.

A system is established where it can over-express these genes in viva A modified doxycycline-inducible lentiviral expression system is used (FIG. 17). Subsequently, each of the selected BMP target genes: BMP6, Gremlin, Bambi, id2 and id3 is cloned into the pEN_T vector under TRE promoter-driven mCherry for the gain of function experiments (FIG. 17). Then the pEN_T vector with the gene of interest (GOI) will be recombined with pSLIK (BSL1, Tet-on) by site-specific recombination (FIG. 17).

The recombined pSLIK vector with TRE promoter-mCherry-IRES-GOI will be used to transduce the hfSCs in vitro (YFP+, KO and Control). Following in vitro doxycycline treatment, FACS sorting of mCherry expressing hfSC populations (low and high fractions) will provide a resource of hfSC populations which possess a range of inducible gene expression abilities. This will be an excellent tool to address the delicate balance of BMP target gene expression needed to maintain hfSC homeostasis.

It is contemplated that BMP signaling directly regulates the target genes: Bmp6, Bambi, Grem1—this is validated for id2, id3 in cultured adult hfSCs. By P-Smad ChIP assays one can expect to specifically immunoprecipitate chromatin-protein complexes that contained sequences that encompassed one of the putative Smad binding sites in the target genes not only in vitro but this example will be able to check these changes in vivo (FIG. 18H).

From the approach with the inducible lentivirus system one expect to efficiently modify hfSCs in vitro and then after reconstitution assay in vivo. The readout for this experiment will be the changes in hfSCs homeostasis, for example normally in the BMP loss of function, hfSCs are permanently activated (no CD34 marker, no quiescence, no visible hair shaft) which this example can easily assess on a grafted skin area. After overexpression, for example the BMP downstream effectors genes id2 or id3 (particularly in BMPR1A KO YFP+ hfSCs) restoration of hfSCs quiescent homeostasis with CD34 will be observed. Therefore, this system allows us to verify biological function of proposed genes much faster in comparison to classical Tg or KO mouse generation.

Sustained BMP Signaling Causes Loss of hfSCs Markers, but hfSCs Persist and can Regenerate New Hair Upon BMP Normalization

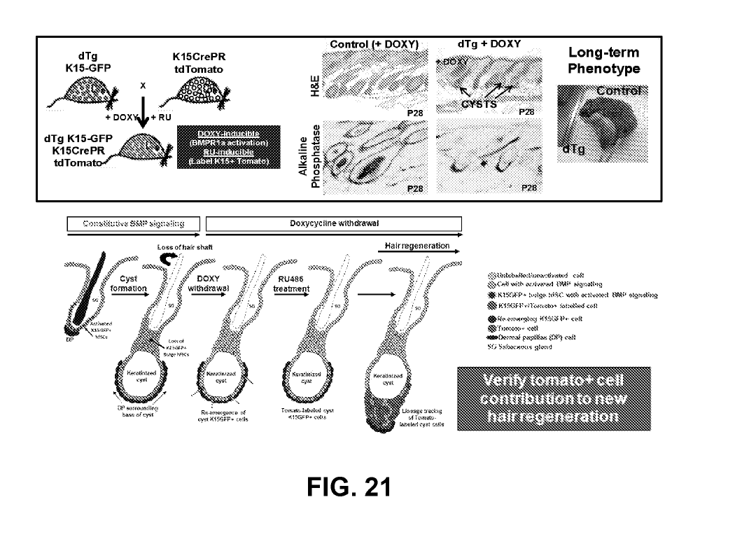

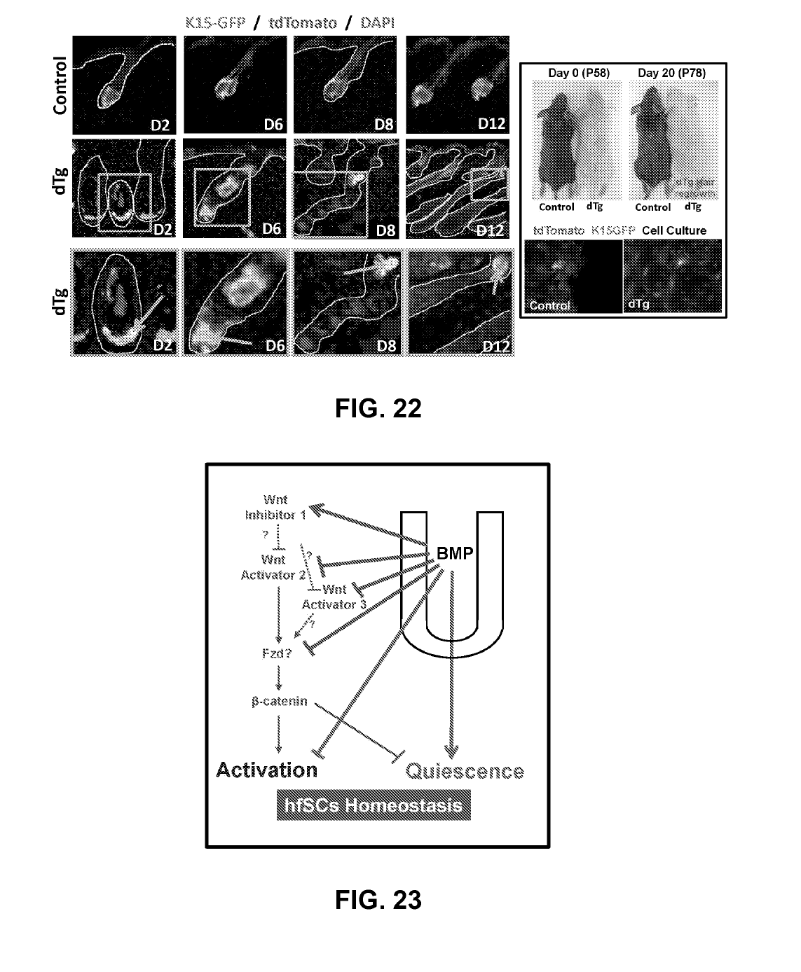

Using this system, it is shown that although sustained BMP signaling results in the loss of hfSCs markers, hfSCs persist and can regenerate new hair. Upon normalization of BMP signaling, however, new hair growth is initiated (FIG. 20). A lineage tracing experiment set-up & plan (FIG. 21) is used to verify tomato+ cell contribution to new hair generation. As shown in FIG. 22, mis-positioned dTg GFP+ cyst cells can initiate new hair growth following BMP normalization. GFP+/tdTomato cyst cells actively participate in new hair formation and can be isolated and cultured in vitro. Therefore, GFP+/tdTomato cyst cells actively participate in new hair formation and can be isolated and cultured in vitro.

Tese parallel loss- and gain-of-function systems which, therefore for the first time, enable the present inventors to specifically mark and isolate live hf SCs after inducible BMP activation or inhibition in vivo. Microarray profiling reveals that YFP+Bmpr1a KO hfSCs down-regulate ˜25% of common up-regulated hfSCs signature genes and adopt some molecular characteristics towards hair germ but still retain multipotent SC characteristics in vitro and in vivo. It is demonstrated that sustained BMP signaling causes loss of hfSCs markers, but hfSCs persist and can regenerate new hair upon BMP normalization (FIG. 23).

### Which Smad Proteins are Important in Hf SC BMP Signaling?

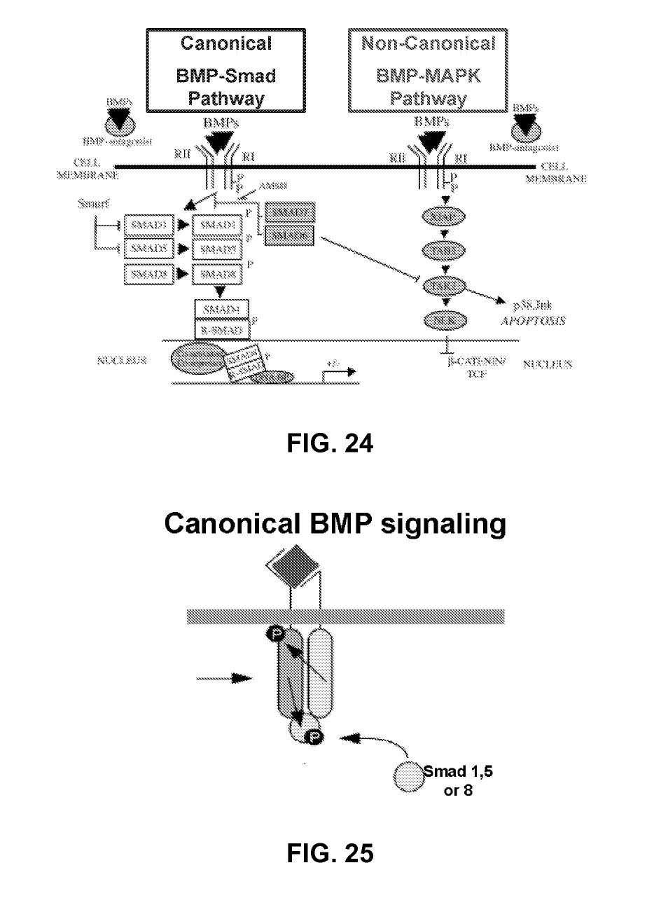

These data also show that in hf SCs, Bmpr1a acts through the Canonical BMP-Smad signaling pathway (FIG. 24). Then, it is important to know which Smad proteins are important in hf SC BMP signaling (FIG. 25). BMP signaling acts via recruitment and phosphorylation of Smads 1, 5 or 8. However, precisely which individual Smad protein or combination of Smad proteins are important in hf SC BMP signaling has not been determined.

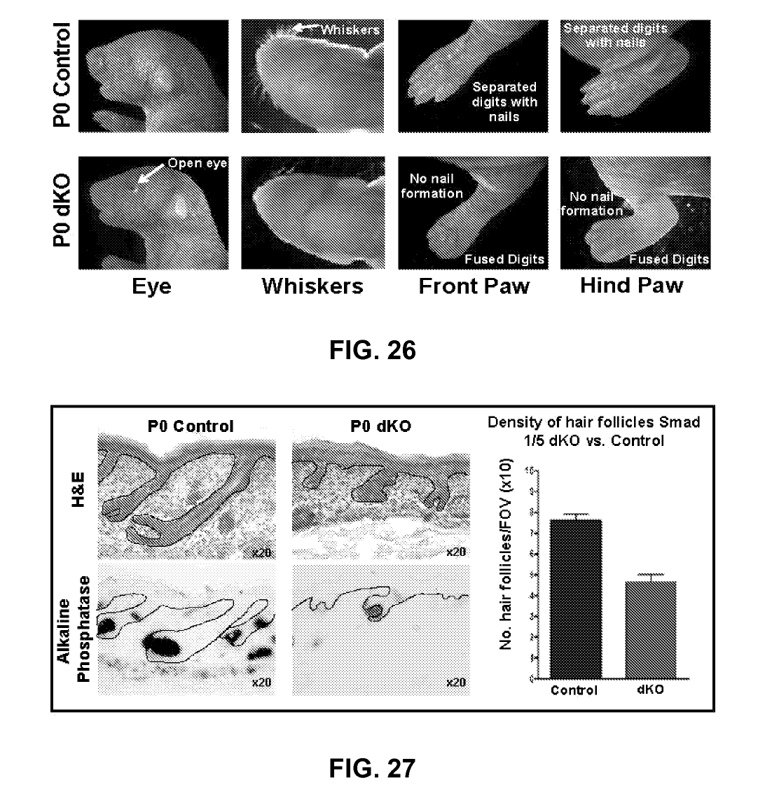

FIG. 26 shows that newborn Smad 1 &5 dKO mice display eye, whisker and paw abnormalities and die within 24 hrs of birth. Smad 1 &5 dKO hair follicles are fewer and under-developed (FIG. 27). Newborn Smad 1&5 dKO skin displays fewer and under-developed hair follicles than control skin.

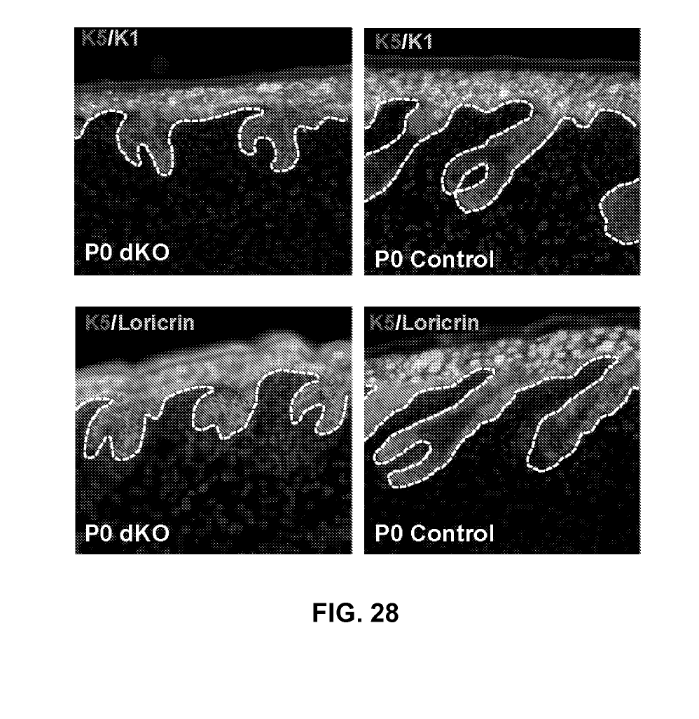

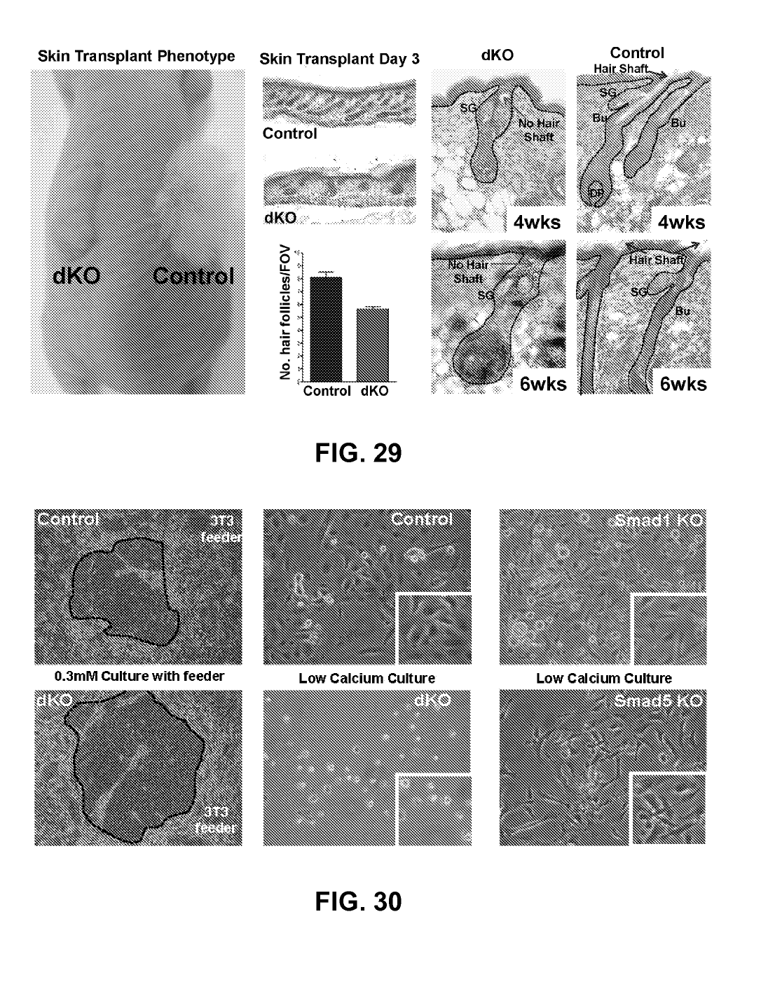

As shown in FIG. 28, skin from Smad 1&5 dKO mice display normal epidermal differentiation but poorly developed (immature) hair follicles. Smad 1&5 dKO skin transplants lack hair shaft formation (FIG. 29). Smad 1 &5 deletion prevents normal hair formation indicating Smad8 acting alone cannot rescue loss-of-hair phenotype.

Further, FIG. 30 show that, in vitro, Smad KO cell lines display altered cell morphology. These Smad cell lines can be used as a tool to help uncover which Smad proteins are significant in hf SC BMP signaling.

### Example 4

This example show additional experiments that investigate the roles of BMP activators, inhibitors and effectors in hair germ cell activation and available results.

As an alternative approach, this example will visualize expression of BMP6, Gremlin and Bambi as well as id2 and id3 during normal physiological hair cycles in hfSCs by in situ hybridization (ISH). Additionally, this example will check how expression of these genes changes in the loss and gain of function models. Additionally, as demonstrated in FIG. 18 H this example will be able to perform Chip-assays in vivo using the FACS sorted YFP positive hfSCs (BMP gain of function model). Additionally, in parallel the inducible lentivirus system will be used for loss of function approaches to reversibly suppress BMP candidate target genes.

Study Direct Regulation of the Wnt Pathway by Cyclic BMP Signaling in hfSCs.

The data suggest a new previously not reported mechanism of hfSCs regulation. It is contemplated that Bmp inhibition directly regulates ligand-receptor dependent Wnt activation (FIG. 9) in hfSCs. Increased stabilization of β-catenin in BMPR1A KO hfCSs was observed with precocious telogenanagen activation.

To determine if the activation of the canonical Wnt pathway was ligand-receptor dependent, the current data show that after the initial BMP inactivation there will be a temporal activation of Wnt7a, Wnt7b and Fzd10 in hfSCs, whereas Dkk3 is suppressed (Table 1, FIG. 9). Consequently, the Wnt pathway and hfSCs will be activated in a cell-autonomous manner.

In fact, after BMP inhibition in hfSCs changes were observed in Wnt ligands-receptors (Table 1) before other Wnt target genes took place (Wnt dependent upregulated genes (brown) in FIG. 5E). Actually, at this early point after BMP inhibition in hfSCs only one gene was observed which overlapped between Wnt and BMP pathways—cyclin B1 (ccnb1; colored brown and underlined in FIG. 5E).

Then as soon as expression of BMP6 takes over again and activates BMP signaling, this will suppress Wnt7a, Wnt7b and Fzd10 in hfSCs whereas Dkk3 will be re-activated (Table 1, FIG. 9), leading to Wnt inhibition and hfSC quiescence (FIG. 9). Thus, this example further tests if cyclic BMP signaling directly regulates the Wnt pathway in hfSCs.

So far any Wnt7a, Wnt7b, Fzd10 and Dkk3 role in hfSCs has not been reported. Additionally, any BMP relationship between Wnt7a, Wnt7b, Fzd10 and Dkk3 has not been reported and, therefore, their function in hfSC regulation and the hfSC cycle is not known.

In addition to the BMP loss-of-function model the current inventors have further developed a gain-of function system. In this inducible double transgenic mouse model this example was able to activate BMP signaling by doxycycline (Doxy) and additionally can visualize any changes in the K15-GFP bulge hfSC population (FIG. 18A-F). FACS sorting allowed us to isolate GFP+ hfSCs, and then culture them for further in vivo and in vitro experiments (FIG. 18G).

For example, this example was able to manipulate BMP activation in vitro after Doxy treatment (FIG. 18G). Since some specific gene regulation could happen only in specific cell types in vivo but cannot be validated in vitro this example successfully developed this approach to facilitate the isolation of GFP+ P-smad+ hfSCs from mice after BMP signaling activation (FIG. 18H).

Thus, the developed mouse model and method will be very advanced and useful to validate Wnt7a, Wnt7b, Fzd10 and DKK3 candidate genes directly responding to BMP signaling by chromatin immunoprecipitation (ChiP) assay using an anti-P-Smad Ab (Cell Signaling). Additionally, this example will validate by QPCR these WNT candidate gene changes during the hair cycle by isolating the hfSCs at different time points during the telogen to anagen transition in controls as well as after BMP activation (P18 to P25).

This example will over-express Wnt7a, Wnt7b, Fzd10 and DKK3 using the lentiviral system in hfSCs and manipulate them in vivo in the chamber graft assay.

From in vitro studies this example is expected to confirm that BMP signaling directly regulates the Wnt target genes. P-Smad ChIP assays are expected to specifically immunoprecipitate sequences that encompass one of the putative Smad binding sites in the WNT target genes in vivo (FIG. 18H).

Over-expression of the Wnt candidates in vivo using the inducible lentivirus system combined with a reconstitution assay in vivo will allow one to see biological changes similar to Wnt gain or loss of function in hfSCs. Especially two types of experiments will be very interesting: over-activation of Wnt7a or Wnt7b on the background of dTg hfSCs with an activated BMP pathway as well as Dkk3 over-expression in the BMPR1A KO. In the first experiment it is expected to see if Wnt overexpression can change the quiescent status of activated BMP hfSCs. The second experiment will determine if activated hfSCs will switch to quiescence after Wnt inhibition.

Spatio-Temporal Visualization of Cyclic Self-Regulation of BMP Signaling in hfSCs In Vivo

Use promoter-reporter constructs to visualize the functional states of hfSC in different stages of normal hair cycle. Within the hair bulge, it is contemplated that hfSCs can fluctuate among different functional states, tilting toward quiescence or activation, depending on the ratio of activators to inhibitors surrounding a particular cell at that moment.

The behavior of each hfSC is regulated stochastically by this the cyclic interactions, and the outcome can differ when the activator/inhibitor landscape is altered by the local microenvironment or global macroenvironment. BMP activity keeps hfSCs in quiescent states and suppression of BMP leads to activated states. It is also found that BMP from the adipocytes act as inhibitors to regulate the ability of telogen hair follicles to enter anagen.

Thus hfSCs can sense BMP within the follicle or from the macroenvironment as a suppressor. Interestingly, subsequent molecular characterization led to the finding that when the BMP pathway is suppressed, Wnt signaling is up regulated (Table 1). Indeed, BMP and Wnt signaling are detected within the bulge at all stages of the hair cycle. Based on these observations, BMPs act as inhibitors of activator signaling.

To put these findings together, it is contemplated that oscillating states, similar to that during development exists in the adult bulge stem cells. Both activator and inhibitor signals undergo continuous changes, feedback interactions and balance (oscillating status).

When the activator/inhibitor ratio of a hfSC reaches a threshold at either end, it can enter activated status and progress into TA status or quiescent status. To test this concept, this example will use promoter-reporter constructs to visualize the functional state status of hfSCs within the hair bulge. This example will then track the activation of these pathways within hfSCs as they progress through the hair cycle, and evaluate the proportion of hfSCs with activated BMP and Wnt signaling pathways at each hair cycle stage (e.g., refractory versus competent telogen). This example will then alter BMP activity quantitatively using transgenic mice, and then follow up the changes of BMP/Wnt activity and hair follicle phenotypes.

Measuring hfSCs Functional States with Reporter Mice

This example will measure activation of BMP and/or Wnt pathway with reporter mice. This example will begin by using Id1-LacZ (BRE-lac1, BRE-lac2) [49], Id1 promoter with green fluorescent protein (BRE:gfp) and p-SMAD immunofluorescence staining to evaluate activity along the BMP signaling pathway and Top-Gal, at-Gal, Axin2-d2EGFP to assess activity along the canonical Wnt signaling pathway. This example will determine the percentage that is positive for these substrates. This example will try to cut thick sections, like a skin strip preparation. This example will try to get the whole bulge population in 3D. This example can also use the fluorogenic substrate fluorescein di-β-D-galactopyranoside (FDG-green, Marker Gene Technologies Inc.) that can allow fluorescence and confocal microscopic analysis as well as FASC sorting.

Evaluate hfSCs Functional States in Hair Cycling Stages of Normal Mice

Readouts from these promoter-reporter constructs will be determined during early (refractory) telogen, late (competent) telogen, early (autonomous) anagen, late (propagating) anagen phases of the hair cycle. For this purpose, 1 cm×2 cm strips along the rostral—caudal axis will be collected from the dorsal skin. Within each strip this example will see progression of the hair wave from quiescent to active status and show representations of the various hair cycle stages to be evaluated.

Serial sections will be made through the skin strips and assembled into 3-dimensional reconstructions. This will enable one to localize the expression of LacZ from each of the promoter-reporters within the hair follicles and determine whether the positive signals for the BMP or Wnt pathways are clustered or widely dispersed within the bulge. In the analysis this example will pay particular attention to the proportion of hfSCs within each hair bulge that are positive for BMP and Wnt signaling at different hair cycle stages. These studies will be performed on wild type C57BL6 mice whose hair wave has been characterized. Statistical analyses will be performed on these results to determine their significance.

hfSCs Functional States in Genetically Modified Mice with a BMP Activity Perturbed

This example will use genetically modify mice with a disrupted BMP signaling pathway. Earlier examples have prepared mice with a constitutively active BMP receptor 1A, a loss of function of BMP receptor 1A and K14-Noggin mice that suppress BMP pathway activity.

It has been seen that BMPR1A-deficient and K14-Noggin mice have a shorter hair cycle. In contrast, mice with constitutive BMP receptors block the activation of the hfSCs. These hfSCs can be reactivated upon removal of BMP. Crossing the promoter-reporter lines with the BMP gain and loss of function genetically modified lines this example can test whether inhibition of BMP activity leads to activation of hfSCs or if activation of BMP signaling pushes hfSCs into a quiescent state. Analysis of these experiments will proceed as described for the wild type lines above.

Ultimately, this study should begin to identify how many hfSCs must be activated before progression from telogen to anagen can commence. This example will use a BMP loss of function model on the K15CrePR+/Rosa26-STOP-eYFP (or alternatively Rosa26-STOP-dtTomato) mouse background to express the fluorescent reporter eYFP and inactivate BMP signaling in a few adult K15 positive hfSCs using a single, low dosage application of RU486 during the second postnatal, synchronized telogen at P43 (composed of refractory versus competent telogen). This will permanently target BMPR1A inactivation and simultaneously label only a few hfSCs per bulge with eYFP (or dtTomato) during the resting phase of the hair cycle.

Then this example will be able to test the hypothesis if cyclic changes happened inside hfSCs by evaluating the functional states of hfSCs in the vicinity of the targeted cells. The random inhibition of BMP signaling in one or two hfSCs/per bulge might lead to a cell-autonomous secretion of Wnts (ie., Wnt7a, Wnt7b) and/or suppression of Wnt antagonists (ie., Dkk3) by the targeted cells. An initial consequence might be the temporal activation of adjacent normal hfSCs (non-genetically targeted) in a non-cell autonomous manner in competent telogen hair follicles. Thus, having this animal on either a Wnt- or BMP-reporter mouse background this example will be able monitor dynamic changes in the direct vicinity of this activated hfSC.

Moreover, using this system this example will be able to trace eYFP expression of these targeted cells and check their commitment during telogen-anagen transition. Thus this example will be able to monitor them during hair cycle progression and check their participation in hf bulge homeostasis, bulge overall integrity and/or their progression into TA status. Additionally, using a gain of function model this example specifically expresses a constitutively active form of BMPR1A in hfSCs to activate canonical BMP signaling in a few bulge cells and then monitor their participation in hfSC homeostasis.

Using Doxy activation this example will test if the adjacent hfSCS will be inhibited by a non-cell autonomous mechanism involving canonical Wnt pathway inhibition via Wnt antagonist secretion (ie., Dkk3) and decreased Wnt expression (i.e., Wnt7a, Wnt7b).

Additionally since this system will be reversible it will allow to re-balance BMP signaling levels (by Doxy withdrawal) to re-establish cyclic self-regulation of BMP signaling in targeted (and possibly neighboring) hfSCs. Finally, the K15-CrePR1/R26YFP or previous K15CrePR1 on Rosa26-loxP-stop-Lacz (R26lacZ) system will allow one to perform lineage tracing analysis in vivo to study the role of cell-autonomous and non cell-autonomous mechanisms of BMP signaling on the overall integrity of the hfSC pool and their homeostasis.

Overall, the gain and loss of BMP function animal models are excellent and unique tools to understand how BMP signaling integrates different regulators into a molecular network capable of cyclic activation of hfSCs. Additionally reversibility of the gain of function model will give us a unique opportunity to understand further hfSC homeostasis by monitoring these dynamic changes in targeted hfSCs and in their direct vicinity.

Use of the promoter-reporter constructs will enable one to visualize expression of Wnt and BMP pathways in hfSCs within the hair bulge and allow us to determine the percentage of cells within the bulge responding to each signaling pathway. It is contemplated that bulge cells can be in 3 possible statuses: oscillating, activated, or quiescent (FIG. 9).

In the quiescent hair follicle population it is expected to see a higher proportion of hfSCs with an activated BMP pathway and suppressed Wnt pathway. In contrast, as cells move toward activation, it is expected to see a lower proportion of cells with an activated BMP pathway and an increased proportion of cells with an activated canonical Wnt signaling pathway. In the oscillating population, it is expected that some hfSCs are activated in both BMP and Wnt but in a quantitatively lower level, kind of like in a “bivalent” state.

This example expects to see more quiescent states in early telogen when hfSCs are refractory to anagen re-entry, but more cells in activated states in late telogen when hfSC are competent to enter anagen. It is expected that the transgenic mice with activated BMP pathway will suppress Wnt signaling and will have a higher proportion of hfSCs with an activated BMP pathway. Transgenic mice with disrupted BMP signaling should have a decreased proportion of hfSCs positive for the BMP pathway and an increased proportion of hfSCs positive for the canonical Wnt signaling pathway, leading to a telogen to anagen transition.

By using the fluorogenic substrate, fluorescein di-β-D-galactopyranoside (FDG-green), this example can do FACS analysis to determine the number of hfSCs expressing each of these pathways. Alternatively, this example will use BREgfp and Axin2-d2EGFP for real time measurements to determine the status of hfSCs. If functional states oscillate faster than the half life of Lac Z, the true status may be obscured. This example expects to use a reporter with a shorter half life. This will allow one to evaluate the functional states with higher time resolution.

Re-Establishment of hfSCs with their Regenerative Potential by Modulating BMP Signaling

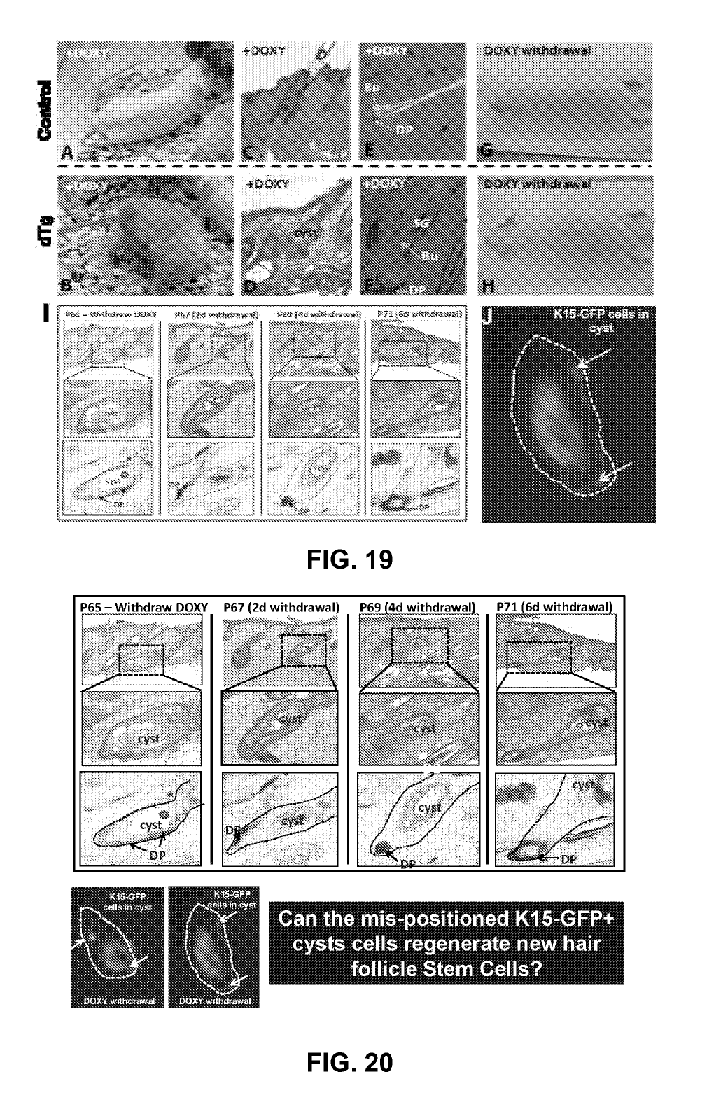

Using the inducible, Bmpr1a gain-of-function system, the earlier examples have demonstrated that constitutive activation of BMP signaling results in inhibition of hfSCs activation and conversion of the hair germ into small non-cycling cysts (FIGS. 19D and C, dTg and control, respectively).

In the proposed model, constant competitive oscillation between activator and inhibitor exists in adult hfSCs and this is critical for maintenance of hfSCs either quiescence or activation (FIG. 9). In this model, molecular characterization revealed that when the BMP pathway is inhibited, Wnt signaling is up regulated (Table 1, FIG. 9).

Conversely it is expected that if BMP will be prolonged activated it would inhibit for extended period of time Wnt signaling and hfSCs activation. However what would be the long term consequences of Wnt prolonged inhibition on hfSCs homeostasis it's not known. To test this concept, this example used promoter-reporter (a K15-GFP) construct to visualize the hfSCs within the hair bulge during prolonged activation of hfSCs.

This approach has been highly advantageous, enabling the monitoring, isolation and characterization of live K15-GFP+ hfSCs following inducible activation of BMP signaling SCs in vivo. It is found that constitutive prolonged activation of BMP signaling promoting progressive hair loss (FIGS. 19B and A, dTg and control, respectively) and loss of biochemical markers of bulge hfSC such as CD34 and K15-GFP, however morphological bulge position remains intact (judged by position of arrector pili muscles) (FIGS. 19F and E, for dTg and control, respectively). Surprisingly, even though some of hfSCs biochemical characteristics are perturbed, after re-balancing the level of BMP signaling, dTg mice were able to re-grow hair (FIG. 19H). Thus suggesting that the hfSC population is able to persist upon prolonged BMP inhibition and can re-establish cyclic regeneration of new hair.

Intriguingly, acutely after DOXY removal (within the 1st week), progressive thickening and downward outgrowth originating from the cysts were observed (FIG. 19I) and, this demonstrated the re-emergence of K15-GFP+ cells within the cyst structures but not in the hf bulge (FIG. 19J). Therefore, this examples specifically targets and labels these re-emerging K15-GFP+ cells (and all progeny) using lineage trace analysis to validate their potential contribution towards new hair formation with a clear goal of furthering the understanding hfSCs regulation and plans to further develop the inducible, gain of function system to encompass a K15CrePR-based lineage tracing feature to specifically target and verify the involvement of mis-positioned hair cyst GFP+ cells in the regeneration of new hair formed following DOXY withdrawal.

This example will cross the dTg-GFP+ (and control GFP+ mice) onto a K15CrePR/Rosa26-STOP-tdTomato background (where the fluorescent reporter tdTomato is permanently expressed by cells which express Cre) to produce dTg-GFP+ and control-GFP+ mice genotypically positive for both K15CrePR1 and tdTomato. By employing RU486 treatment, this example can then induce tdTomato expression in K15+ cells and thus permanently mark the re-emerging K15-GFP+ cyst cells (and their progeny) to monitor (by tdTomato expression) their role in new hair growth following DOXY removal (2 to 4 days).

At each point, when this example collects skin samples for analysis this example will also check the proliferation status of these cells (pulsing animals with BrdU for 2 to 3 h before sample collection). Using FACS sorting this example will isolate and characterize the re-emerging tdTomato+ cells during the early re-organization events occurring during new hair formation.

It is contemplated that, following RU486 treatment, this example can specifically target and permanently label re-emerging K15-GFP+ hair cyst cells (and progeny) by tdTomato. It is expected K15-GFP+ expression will be lost by some cells which commit to differentiate (i.e. lose hfSCs K15+ expression) and promote hair shaft formation during new hair regeneration. These cells will be permanently marked by tdTomato and, consequently, this example will be able to trace them in new hair formation.

In addition, it is expected some cells to remain positive for both K15-GFP+ and tdTomato (potential new hfSC pool) and propose that these cells may be responsible for reforming or repopulating the new hfSC bulge.

These studies will further the understanding of hfSC regulation and how they achieve and maintain regenerative potential during re-establishment the hfSC homeostasis. As an alternative approach, in place of using fluorescent reporter Rosa26-STOP-tdTomato mice, this example plan to cross the gain-of-function model onto a K15CrePR/Rosa26LacZ background and use the same K15-based approach to target the re-emerging K15-GFP+ cell population. This system will be advantageous if, during histological analysis, the background fluorescence in the tdTomato+ samples is high and this example can instead use Xgal staining to monitor hair cyst GFP+ cell (and progeny) contributions towards new hair production.

Additional, follow on studies have subsequently been conducted and the results are attached to this document, and incorporated by reference it their entirety.

Moreover, the versatility of the lineage tracing approach will be advantageous in that one can activate BMP signaling and/or label K15+ cells at the convenience, i.e. during initial activation of BMP signaling by DOXY (to mark K15+ hfSCs before hf cyst formation) to evaluate their contribution to the cyst structures or after DOXY withdrawal to mark the contribution towards new hair regeneration as proposed above. Actually, initially labeled cells by dtTomato in this alternative approach would be FACS sorted during quiescence state upon Doxy treatment and BMP activation. Thus this example would learn what the molecular characteristic of these cells is when they are quiescent and when other biochemical markers of hfSC such as CD34 and K15-GFP are no longer expressed. Then upon Doxy withdrawal when this example re-inhibit BMP signaling it will re-activate K15-GFP expression (FIG. 19J) in the cells already marked by tdTomato+ thus this example will able to isolate double positive fraction of cells re-emerging from this cyst at the onset of new hair formation.

It should be noted that although the discussions herein may refer to a specific order and composition of method steps, it is understood that the order of these steps may differ from what is described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present invention. Such variations will depend on the software and hardware systems chosen and on designer choice. It is understood that all such variations are within the scope of the invention. Likewise, software and web implementations of the present invention could be accomplished with standard programming techniques with rule based logic and other logic to accomplish the various database searching steps, correlation steps, comparison steps and decision steps.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. An isolated hair follicle stem cell (hfSC) that expresses keratin 15 (K15) and has decreased BMP signaling.

2. The isolated hfSC of claim 1, wherein the hfSC has decreased biological activity of BMPR1A.

3. The isolated hfSC of claim 2, wherein the BMPR1A is inactivated.

4. The isolated hfSC of claim 1, wherein the hfSC does not express CD34.

5. The isolated hfSC of claim 1, further comprising an exogenous reporter gene.

6. The isolated hfSC of claim 5, wherein the reporter gene is activated by a K15 promoter.

7. The isolated hfSC of claim 6, wherein the reporter gene is activated by a gene downstream of the K15 promoter.

8. The isolated hfSC of claim 7, wherein the activation is mediated by a pharmaceutical agent that is in contact with the hfSC.

9. The isolated hfSC of claim 1, wherein the hfSC has increased expression of one or more of Wnt7a, Wnt7b, or Fzd10 and/or decreased expression of Dkk3 each as compared to a hfSC having normal BMP signaling.

10. A population of isolated hfSC of claim 1.

11. The population of claim 9, wherein the population is substantially homogenous.

12. A substantially homogenous population of cells differentiated from an isolated hfSC of claim 1.

13. A pharmaceutical composition comprising an isolated hfSC of claim 1, and a pharmaceutically acceptable carrier.

14. A method of treating alopecia in a mammalian subject, comprising implanting to the subject an isolated hair follicle stem cell (hfSC) of claim 1.

15. An isolated hair follicle stem cell (hfSC) comprising a constitutively active BMPR1A gene.

16. The isolated hfSC of claim 15, wherein the BMPR1A gene is regulated by a promoter inducible by a pharmaceutical agent that is in contact with the hfSC.

17. The isolated hfSC of claim 16, wherein the promoter is double Tg (dTg).

18. The isolated hfSC of claim 17, wherein the pharmaceutical agent is doxcycline.

19. The isolated hfSC of claim 15, further comprising a reporter gene regulated by a keratin 15 (K15) promoter.

20. A population of isolated hfSC of claim 15.

21. The population of claim 20, wherein the population is substantially homogenous.

22. A clonal population of isolated hfSC of claim 15.

23. A transgenic non-human mammal comprising a hair follicle stem cell (hfSC) comprising a constitutively active BMPR1A gene.

24. The genetically engineered non-human mammal of claim 23, wherein the hfSC further comprises a reporter gene regulated by a keratin 15 (K15) promoter.

25. A method of identifying an agent suitable for inhibiting BMP signaling in a hair follicle, the method comprising contacting a candidate agent with an isolated hfSC of claim 19, wherein expression of the reporter gene indicates that the candidate agent is suitable for inhibiting BMP signaling in a hair follicle.

26. A method of identifying an agent suitable for activating a hair follicle stem cell (hfSC), the method comprising contacting a candidate agent with an isolated hfSC of claim 19, wherein expression of the reporter gene indicates that the candidate agent is suitable for activating a hfSC.

27. A method of activating a hair follicle stem cell (hfSC), comprising increasing the biological activity of one or more of Wnt7a, Wnt7b, or Fzd10 and/or decreasing the biological activity of Dkk3.

28. The method of claim 27, wherein the increasing of the biological activity of one or more of Wnt7a, Wnt7b, or Fzd10 comprises increasing of transcription of one or more of Wnt7a, Wnt7b, or Fzd10.

29. The method of claim 27, wherein the decreasing of the biological activity of Dkk3 comprises decreasing the transcription of Dkk3.

30. The method of claim 27, further comprising contacting the hfSC with an agent that inhibits BMP signaling.

31. A method of treating alopecia in a mammalian subject, comprising implanting to the subject an activated a hair follicle stem cell (hfSC), wherein the hfSC was isolated from the subject and activated by a method of claim 27.
